(19) United States
(12) Patent Application Publication
(10) Pub. No.: US 2016/0249870 A1
(43) Pub. Date: Sep. 1, 2016

(54) RADIATION IMAGING SYSTEM, IMAGING STAND, AND IMAGING METHOD

(71) Applicant: FUJIFILM Corporation, Tokyo (JP)

(72) Inventor: Takashi Tajima, Ashigarakami-gun (JP)

(73) Assignee: FUJIFILM Corporation, Tokyo (JP)

(21) Appl. No.: 14/985,545

(22) Filed: Dec. 31, 2015

(30) Foreign Application Priority Data
Feb. 26, 2015 (JP) 2015-037419

(51) Int. Cl. A61B 6/00; A61B 6/10

(52) U.S. Cl. CPC A61B 6/4291; A61B 6/107; A61B 6/4266; A61B 6/46; A61B 6/4283

(57) ABSTRACT

It is possible to improve convenience for a user. A radiation imaging system includes radiation detectors which have an imaging surface, on which radiation is incident, and generate a radiation image of an imaging target according to radiation transmitted through the imaging region as an imaging target of an object and incident on the imaging surface, a grid for normal imaging which has an effective area narrower than the imaging surface and eliminates scattered radiation included in radiation transmitted through the imaging target and incident on the effective area, and a moving unit which functions as a holding unit capable of holding the grid for normal imaging at a plurality of positions along the imaging surface where the imaging surface and the effective area overlap each other.

### CROSS-REFERENCE TO RELATED APPLICATIONS

The present application claims priority under 35 U.S.C. §119 to Japanese Patent Application No. 2015-037419, filed on Feb. 26, 2015. The above application(s) is hereby expressly incorporated by reference, in its entirety, into the present application.

### BACKGROUND OF THE INVENTION

1. Field of the Invention

The present invention relates to a radiation imaging system, an imaging stand, and an imaging method.

2. Description of the Related Art

Hitherto, as a radiation imaging device which images a radiation image of an object, a radiation imaging device which performs imaging for the purpose of medical diagnosis has been known. The radiation imaging device detects radiation irradiated from a radiation irradiation device and transmitted through the object with a radiation detector to generate a radiation image.

In this type of radiation imaging device, there is a case where a comparatively large imaging target, for example, an elongated imaging target, such as a spine or a lower limb, is imaged. As a technique for imaging an elongated imaging target, for example, a technique described in JP2002-301055A is known. JP2002-301055A describes a technique for generating a complete composite image from two partial images with no artifact in elongated imaging.

### SUMMARY OF THE INVENTION

In general, in imaging a radiation image, if radiation is transmitted through an imaging target, scattered radiation is generated according to a component of the imaging target; thus, scattered radiation is included in radiation transmitted through the imaging target. For this reason, a grid which eliminates scattered radiation is provided between the imaging target and the radiation detector, thereby eliminating scattered radiation included in radiation reaching the radiation detector. Scattered radiation is eliminated by the grid, whereby degradation of contrast of the radiation image is suppressed, and image quality of the radiation image is improved.

Elimination of scattered radiation is performed, for example, even in an elongated imaging target regardless of the size of the imaging target. Furthermore, as the grid which eliminates scattered radiation, a grid of a type according to an imaging method or the amount of scattered radiation is used. For this reason, a grid of a type according to the size of the imaging target, the amount of scattered radiation, or the like should be prepared, and accordingly, convenience for a user may be poor.

An object of the invention is to provide a radiation imaging system, an imaging stand, and an imaging method capable of improving convenience for a user.

In order to attain the above-described object, a radiation imaging system of the invention includes a radiation detector which has an imaging surface, on which radiation is incident, and generates a radiation image of an imaging target according to radiation transmitted through the imaging target and incident on the imaging surface, a grid which has an effective area narrower than the imaging surface and eliminates scattered radiation included in radiation transmitted through the imaging target and incident on the effective area, and a holding unit which is able to hold the grid at a plurality of positions along the imaging surface where the imaging surface and the effective area overlap each other.

A radiation imaging system of the invention includes a radiation detector group which includes a plurality of radiation detectors configured to have an imaging surface, on which radiation is incident, and to generate a radiation image of an imaging target according to radiation transmitted through the imaging target and incident on the imaging surface, adjacent radiation detectors being arranged in parallel in a direction intersecting an incidence direction of radiation and the end portions of the imaging surfaces of adjacent radiation detectors overlapping each other in the incidence direction of radiation, a grid which has an effective area narrower than the imaging surface by the radiation detector group and eliminates scattered radiation included in radiation transmitted through the imaging target and incident on the effective area, and a holding unit which is able to hold the grid at a plurality of positions along the imaging surface where a first area where the end portions of adjacent radiation detectors overlap each other and the end portion of the grid are separated in the intersection direction, and the imaging surface and the effective area overlap each other.

The radiation imaging system of the invention may further include a warning unit which performs a warning determined in advance in the case where the end portion of the grid is held at a position overlapping the first area in the intersection direction.

A radiation imaging system of the invention includes a radiation detector group which includes a plurality of radiation detectors configured to have an imaging surface, on which radiation is incident, and to generate a radiation image of an imaging target according to radiation transmitted through the imaging target and incident on the imaging surface, adjacent radiation detectors being arranged in parallel in a direction intersecting an incidence direction of radiation and the end portions of the imaging surfaces of adjacent radiation detectors overlapping each other in the incidence direction of radiation, a grid which has an effective area narrower than the imaging surface by the radiation detector group and eliminates scattered radiation included in radiation transmitted through the imaging target and incident on the effective area, and a holding unit which is able to hold the grid at a plurality of positions along the imaging surface where a second area in the radiation images generated by the radiation detector group where a step image due to a step with respect to the incidence direction in the end portions of the radiation detectors is included and an end image due to the end portion of the grid are separated, and the imaging surface and the effective area overlap each other.

The radiation imaging system of the invention may further include a warning unit which performs a warning determined in advance in the case where the grid is held at a position in the second area where the end image is included.

In the radiation imaging system of the invention, the position of the second area and the position of the end image may be determined based on the incidence angles of radiation with respect to the grid and the radiation detectors.

In the radiation imaging system of the invention, the holding unit may include a moving unit which is able to move the grid in a direction along the imaging surface.

The radiation imaging system of the invention may further include a reception unit which receives an instruction to move the grid, and the moving unit may move the grid based on the instruction received by the reception unit.

In the radiation imaging system of the invention, the holding unit may include a control unit which controls the moving of the grid by the moving unit.

The radiation imaging system of the invention may further include an imaging stand which has the holding unit and a shield unit capable of shielding the grid from the imaging target, and the control unit may move the grid with the moving unit in the case where the shield unit shields the grid from the imaging target.

The radiation imaging system of the invention may further include a radiation irradiation device which irradiates radiation, and the radiation irradiation device may move in a direction intersecting the incidence direction of radiation in conjunction with the moving of the grid by the moving unit.

An imaging stand of the invention includes a housing unit which houses a radiation detector configured to have an imaging surface, on which radiation is incident, and to generate a radiation image of an imaging target according to radiation transmitted through the imaging target and incident on the imaging surface, and a grid configured to have an effective area narrower than the imaging surface and to eliminate scattered radiation included in radiation transmitted through the imaging target and incident on the effective area, and a holding unit which is able to hold the grid at a plurality of positions along the imaging surface where the imaging surface and the effective area overlap each other.

In the imaging stand of the invention, the holding unit may include a moving unit which is able to move the grid in a direction along the imaging surface.

An imaging method of the invention includes, in the case where a radiation image is imaged using a radiation detector group which includes a plurality of radiation detectors configured to have an imaging surface, on which radiation is incident, and to generate a radiation image of an imaging target according to radiation transmitted through the imaging target and incident on the imaging surface, adjacent radiation detectors being arranged in parallel in a direction intersecting an incidence direction of radiation and the end portions of the imaging surfaces of adjacent radiation detectors overlapping each other in the incidence direction of radiation, and a grid which has an effective area narrower than the imaging surface by the radiation detector group and eliminates scattered radiation included in radiation transmitted through the imaging target and incident on the effective area, a step of causing a holding unit capable of holding the grid to hold the grid at a plurality of positions along the imaging surface where a first area where the end portions of adjacent radiation detectors overlap each other and the end portion of the grid are separated in the intersection direction, and the imaging surface and the effective area overlap each other, and causing a control unit to perform moving control for moving the grid to any of the plurality of positions by a moving unit capable of moving the grid in a direction along the imaging surface.

An imaging method of the invention includes, in the case where a radiation image is imaged using a radiation detector group which includes a plurality of radiation detectors configured to have an imaging surface, on which radiation is incident, and to generate a radiation image of an imaging target according to radiation transmitted through the imaging target and incident on the imaging surface, adjacent radiation detectors being arranged in parallel in a direction intersecting an incidence direction of radiation and the end portions of the imaging surfaces of adjacent radiation detectors overlapping each other in the incidence direction of radiation, and a grid which has an effective area narrower than the imaging surface by the radiation detector group and eliminates scattered radiation included in radiation transmitted through the imaging target and incident on the effective area, a step of causing a holding unit capable of holding the grid to hold the grid at a plurality of positions along the imaging surface where an end image due to the end portion of the grid is not included in a second area in the radiation images generated by the radiation detector group where a step image due to a step with respect to the incidence direction in the end portions of the radiation detectors is included, and the imaging surface and the effective area overlap each other, and causing a control unit to perform moving control for moving the grid to any of the plurality of positions by a moving unit capable of moving the grid in a direction along the imaging surface.

According to the invention, it is possible to improve convenience for the user.

### BRIEF DESCRIPTION OF THE DRAWINGS

FIG. 1 is a schematic configuration diagram showing an example of a radiation imaging system of an embodiment.

FIG. 2 is a perspective view of an imaging stand of a first embodiment when viewed from the side.

FIGS. 3A and 3B are a front view and a side view of the imaging stand of the first embodiment when viewed from an irradiation side of radiation.

FIG. 4 is a block diagram showing an example of the schematic configuration of a radiation imaging device, an imaging stand, and a console of a radiation imaging system of the first embodiment.

FIG. 5 is a flowchart showing an example of the flow of a grid moving process which is executed by a control unit of the console of the first embodiment.

FIGS. 6A to 6C are explanatory views illustrating the positions of a radiation irradiation device, a radiation imaging device, and a grid for normal imaging of the embodiment and an irradiation state of radiation.

FIG. 7 is a partial enlarged view of a portion of an avoidance area of the grid for normal imaging in FIGS. 6A to 6C.

FIG. 8 is a perspective view of an imaging stand of a second embodiment when viewed from the side.

FIG. 9 is a block diagram showing an example of the schematic configuration of a radiation imaging device, an imaging stand, and a console of a radiation imaging system of the second embodiment.

FIG. 10 is a flowchart showing an example of the flow of a grid moving process which is executed by a control unit of the console of the second embodiment.

FIGS. 11A to 11C are a front view and a side view of an imaging stand of a third embodiment when viewed from an irradiation side of radiation.

FIG. 12 is a block diagram showing an example of the schematic configuration of a radiation imaging device, an imaging stand, and a console of a radiation imaging system of the third embodiment.

FIG. 13 is a flowchart showing an example of the flow of a grid holding process which is executed by a control unit of the console of the third embodiment.

FIG. 14 is a flowchart showing an example of the flow of a grid moving process which is executed by a control unit of a console of a fourth embodiment.

FIGS. 15A and 15B are a side view of radiation detectors when the radiation detectors are arranged in parallel while aligning the height of an imaging surface and a front view of radiation detectors when viewed from an irradiation side of radiation R.

FIG. 16 is a side view when a radiation imaging system includes a plurality of radiation imaging devices having one radiation detector.

FIG. 17 is a block diagram showing an example of the schematic configuration of a radiation imaging system including a portable information terminal device.

### DESCRIPTION OF THE PREFERRED EMBODIMENTS

Hereinafter, an example of an embodiment according to the invention will be described referring to the drawings. In the respective drawings, portions having the same functions are represented by the same reference numerals, and overlapping description will be appropriately omitted.

### First Embodiment

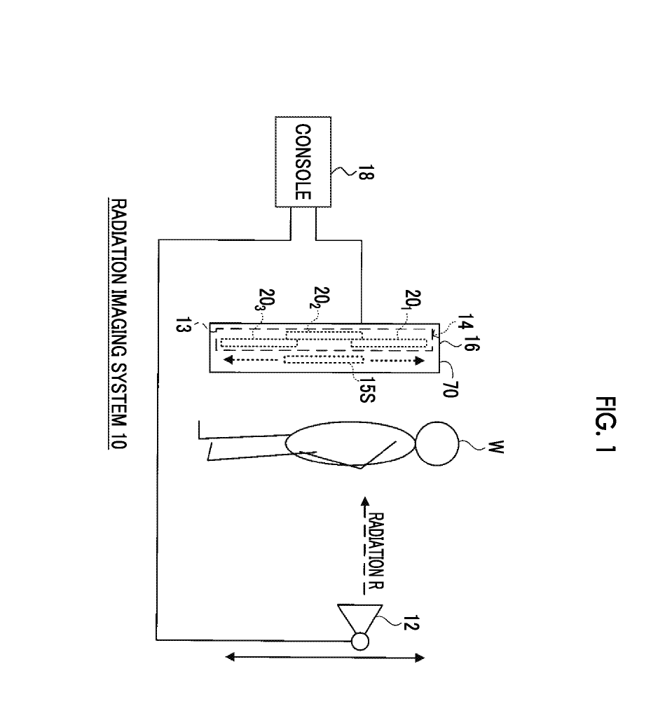

First, the schematic configuration of a radiation imaging system of this embodiment will be described. FIG. 1 is a schematic configuration diagram showing an example of the radiation imaging system of this embodiment.

The radiation imaging system 10 of this embodiment includes a radiation irradiation device 12, a radiation imaging device 14, an imaging stand 16, and a console 18.

The radiation irradiation device 12 includes a radiation source (not shown). The radiation irradiation device 12 has a function of irradiating an object W with radiation X (for example, X-rays or the like) from the radiation source. While a method of instructing the radiation irradiation device 12 to execute irradiation of radiation R is not particularly limited, in this embodiment, an execution instruction of irradiation is performed from the console 18. In this embodiment, the radiation irradiation device 12 is arranged to be movable in the longitudinal direction (the up-down direction of FIG. 1) of the radiation imaging device 14. In the following description, the position of a bulb actually irradiating radiation R is aligned with the position of the radiation irradiation device 12. The longitudinal direction of the radiation imaging device 14 of this embodiment is an example of a direction intersecting an incidence direction of radiation R of the invention.

The radiation imaging device 14 includes, in a housing 13, a plurality of (in this embodiment, three) radiation detectors 20₁ to 20₃ which detect radiation R irradiated from the radiation irradiation device 12 and transmitted through the object W to generate image data of a radiation image. A plurality of radiation detectors 20₁ to 20₃ of this embodiment are an example of a radiation detector group of the invention. Hereinafter, when the radiation detectors 20₁ to 20₃ are collectively referred to, the reference numeral at the end distinctively indicating each radiation detector is omitted, and the radiation detectors are referred to as the “radiation detectors 20”. The number of radiation detectors 20 is not limited to the number in this embodiment.

The radiation imaging device 14 has a function of imaging the radiation image of the object W by generating image data of the radiation image using the radiation detectors 20. In this embodiment, as the radiation imaging device 14, an electronic cassette for elongated imaging is used.

In the radiation imaging device 14 of this embodiment, as shown in FIG. 1, the end portion of the radiation detector 20 is arranged to overlap the end portion of the adjacent radiation detector 20 (the details will be described below).

The radiation imaging device 14 has a plurality of radiation detectors 20 arranged in the above-described manner, and the radiation imaging device 14 has an elongated imaging surface as a whole.

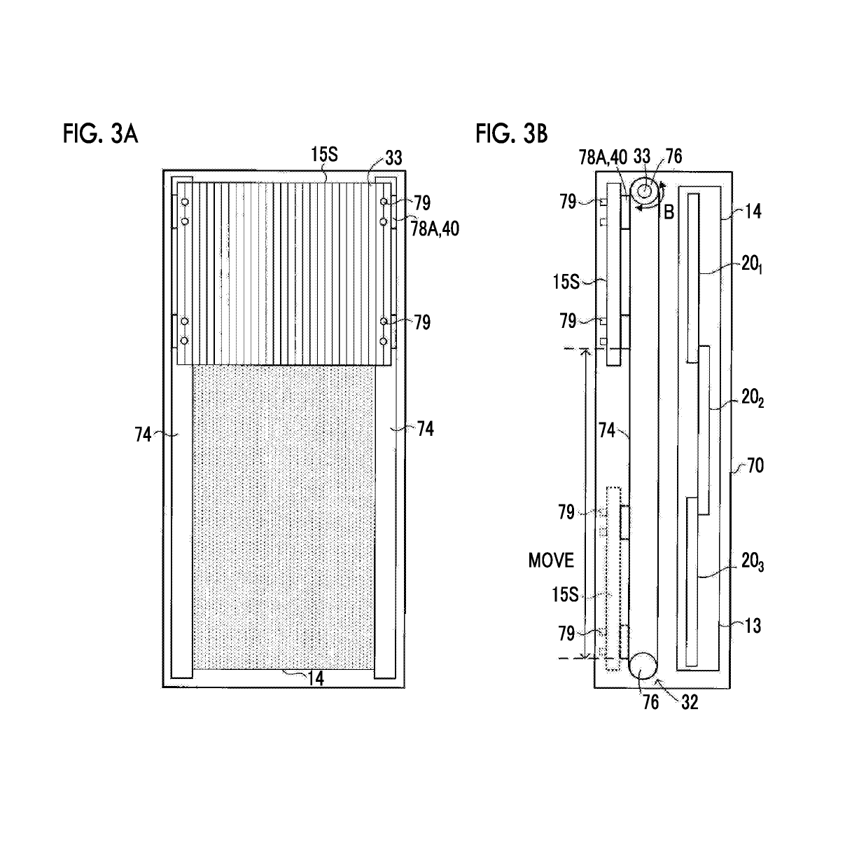

The imaging stand 16 of the embodiment has a function of housing the radiation imaging device 14 and a grid for elongated imaging (not shown) or a grid 15S for normal imaging when imaging is performed. The imaging stand 16 of this embodiment includes a moving unit 32 (not shown in FIG. 1, see FIGS. 3A and 3B), has a function of holding the grid 15S for normal imaging to be movable in the up-down direction of FIG. 1 using the moving unit 32.

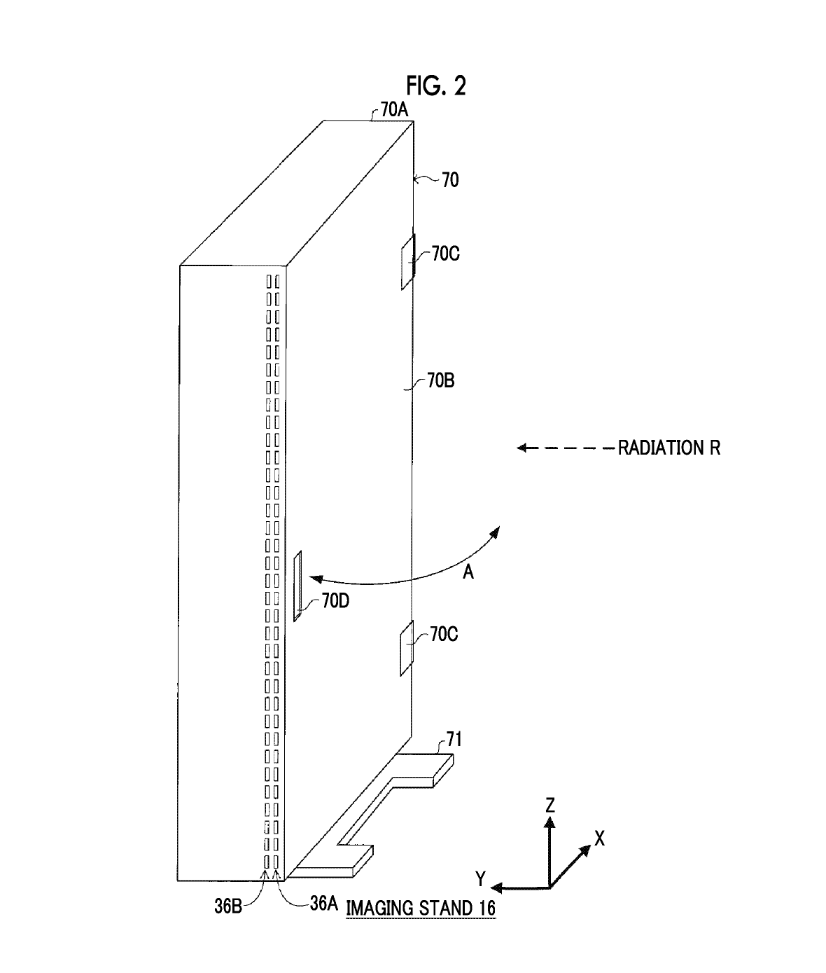

FIG. 2 is a perspective view of the imaging stand 16 of this embodiment when viewed from the side. As shown in FIG. 2, hereinafter, description will be provided assuming that the right-left direction of the imaging stand 16 in front view is the X-axis direction, the depth direction of the imaging stand 16 in front view is the Y-axis direction, and the up-down direction of the imaging stand 16 in front view is the Z-axis direction.

As shown in FIG. 2, the imaging stand 16 includes a housing 70, of which the longitudinal direction is the Z-axis direction and the lateral direction is the X-axis direction. In a lower end portion of the housing 70, a bottom plate 71 is provided, and the housing 70 is stably installed by the bottom plate 71.

In the housing 70, a door 70B which is an example of a shield unit of the invention is attached to a main body 70A having a substantially rectangular parallelepiped shape by hinges 70C to be openable in an arrow A direction. The door 70B is provided with a grip portion 70D that a user, such as a physician or a radiologist, grips to open or close the door 70B. Furthermore, a positioning recommended position display unit 36A and a grid position display unit 36B are provided on a pair of side surfaces of the main body 70A. In FIG. 2, though not shown, the same positioning recommended position display unit 36A and the grid position display unit 36B as those shown in the drawing are also provided on the side surface in the X-axis direction.

When a radiation image is imaged, the side on which the door 70B of the housing 70 becomes the irradiation side of radiation R, and the radiation imaging device 14 is irradiated with radiation R transmitted through the object W through the door 70B and the grid 15S for normal imaging or the grid for elongated imaging (not shown) housed in the housing 70.

FIGS. 3A and 3B are a front view and a side view of the inside of the imaging stand 16 when the radiation imaging device 14 of this embodiment and the grid 15S for normal imaging are housed when viewed from the irradiation side of radiation R. FIG. 3A is a front view when the radiation imaging device 14 and the grid 15S for normal imaging are housed. FIG. 3B is a side view when the radiation imaging device 14 and the grid 15S for normal imaging are housed.

In the radiation imaging system 10 of this embodiment, the radiation imaging device 14 is used for both normal imaging where one radiation image is generated only using one radiation detector 20 and elongated imaging where one radiation image is generated using a plurality of radiation detectors 20 for an imaging region larger than normal imaging. In this embodiment, the “imaging region” is a region which is required for reading the radiation image, and refers to at least a region including a region of interest (ROI).

In this embodiment, normal imaging refers to imaging equivalent to when one electronic cassette of a size (for example, 35 cm×43 cm, 43 cm×43 cm, or the like) for general-purpose use in chest imaging, abdomen imaging, and bone imaging is used. Even if all radiation detectors 20 generate image data of the radiation image with single irradiation (so-called one shot) of radiation R, when the number of radiation detectors 20 which generate image data of the radiation image reflecting the imaging region of the object W is equal to or less than two, this is regarded as normal imaging.

In this embodiment, elongated imaging refers to imaging when an imaging region larger than normal imaging of the object W is imaged, for example, spine imaging and lower limb imaging.

In the radiation imaging system 10 of this embodiment, when the radiation image is imaged, radiation R is transmitted through the object W, whereby scattered radiation is generated; thus, the grid for elongated imaging or the grid 15S for normal imaging which has a function of eliminating scattered radiation is used. Hereinafter, when the grid for elongated imaging and the grid 15S for normal imaging are collectively referred to, these are simply referred to as the “grids 15”. Since the amount (predicted amount) of scattered radiation to be generated from the object W is different according to the irradiation amount of radiation R, the grid 15 of a type according to the amount of scattered radiation to be generated is used.

In general, the grid 15 is configured such that a thin film of metal, such as lead having high absorption of radiation R and an intermediate substance (a so-called inter-space) having low absorption of radiation R between the thin films are alternately arranged with fine lattice density. In order for radiation R to be transmitted through the intermediate substance, as the material for the intermediate substance, a material which allows easy transmission of radiation R is preferably used, and for example, aluminum, paper, carbon fiber, or the like is used. In the grid 15, lattice density, the material of the intermediate material, or the like is different according to the amount of scattered radiation to be generated (the amount of scattered radiation to be eliminated) or the like. In this embodiment, an area of the grid 15 where scattered radiation can be eliminated is referred to as an “effective area”.

As shown in FIGS. 3A and 3B, the imaging stand 16 of this embodiment includes the moving unit 32 which moves the grid 15S for normal imaging in the longitudinal direction of the imaging stand 16 (the radiation imaging device 14). The moving unit 32 of this embodiment is an example of a holding unit and a moving unit of the invention. The moving unit 32 has a pair of stepping motors 33, a belt 74, two rollers 76, and two holding stands 78A. The configuration of the moving unit 32 is not limited to this embodiment. For example, a pair of stepping motors 33 may not be provided, and either stepping motor may be provided. For example, ball screws may be used, and as a specific example of a configuration in this case, a configuration in which a screw shaft of the ball screws extending in the longitudinal direction of the imaging stand 16 is provided, and the holding stands 78A are moved with the rotation of the ball screws is considered.

On the surface of the belt 74 extending in the longitudinal direction of the imaging stand 16, the holding stands 78A which hold the grid 15 are fixed. The belt 74 is stretched between a pair of rollers 76, and one roller 76 is rotated in an arrow B direction by driving of the stepping motors 33. The holding stands 78A are moved in the longitudinal direction of the imaging stand 16 with the rotation of the rollers 76.

As shown in FIG. 3A, when normal imaging is performed, the grid 15S for normal imaging is held to be movable to a position covering a part of the radiation imaging device 14 between the radiation imaging device 14 and the object W in the longitudinal direction of the imaging stand 16 by the moving unit 32. Specifically, the grid 15S for normal imaging is fixed to the holding stands 78A by screws 79 and held to be movable in the longitudinal direction of the imaging stand 16 by the moving unit 32.

The console 18 of this embodiment has, for example, a function of controlling the entire radiation imaging system 10 based on order information input from an external system, such as a radiology information system (RIS), or a function of controlling the generation of the radiation image by the radiation imaging device 14. For this reason, the console 18 receives the order information from the external system.

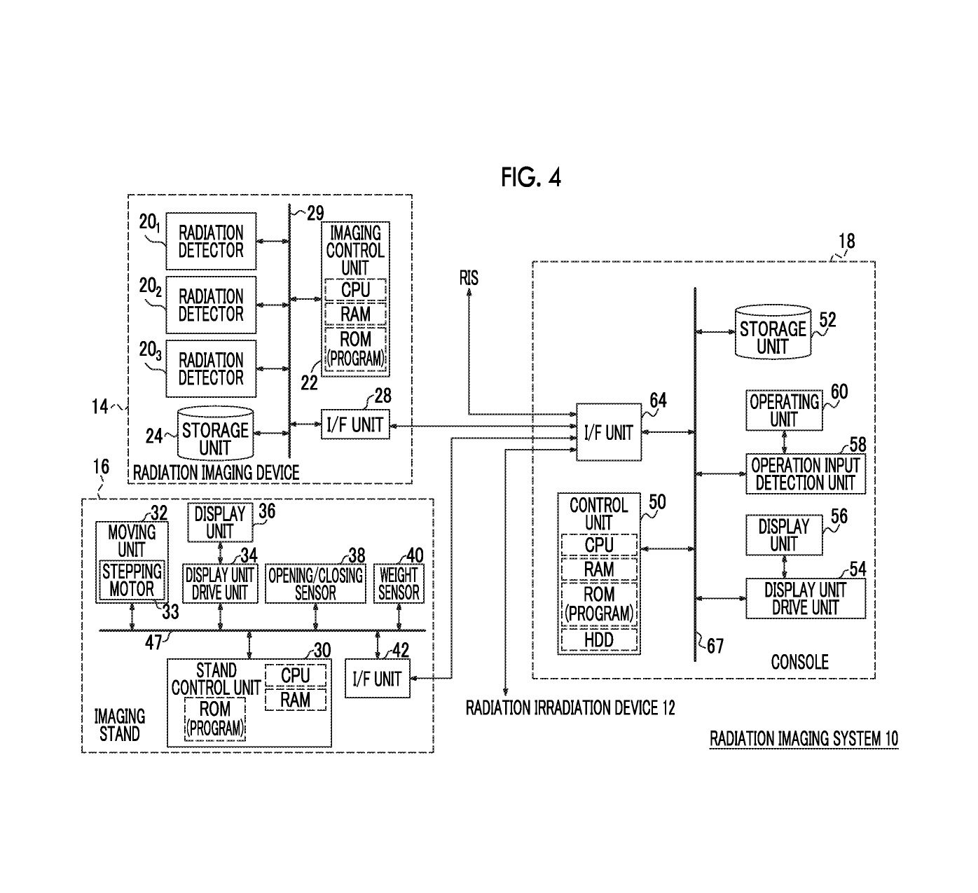

Next, the configurations and functions of the radiation imaging device 14, the imaging stand 16, and the console 18 will be described in detail. FIG. 4 is a block diagram showing an example of the schematic configuration of the radiation imaging device 14, the imaging stand 16, and the console 18 of the radiation imaging system 10 of this embodiment.

The radiation imaging device 14 of this embodiment includes an imaging control unit 22, a storage unit 24, and an interface (I/F) unit 28, in addition to the three radiation detectors 20 described above. The radiation detectors 20, the imaging control unit 22, the storage unit 24, and the I/F unit 28 are connected to transmit and receive various kinds of information through a bus 29, such as a system bus or a control bus, to and from one another.

The radiation detectors 20 have a function of detecting radiation R transmitted through the object W under the control of the imaging control unit 22. The radiation detectors 20 of this embodiment are not particularly limited, and may be indirect conversion type radiation detectors which convert incident radiation R to light and convert converted light to an electric charge, or direct conversion type radiation detectors which directly convert radiation R to an electric charge.

The imaging control unit 22 has a function of controlling the overall operation of the radiation imaging device 14.

The imaging control unit 22 includes a central processing unit (CPU), a read only memory (ROM), and a random access memory (RAM). The ROM stores various processing programs, which are executed on the CPU, in advance. The RAM has a function of temporarily storing various kinds of data.

The storage unit 24 stores image data of the generated radiation image, or the like. As a specific example of the storage unit 24, a solid state drive (SSD) or the like is considered. The storage unit 24 may be integrated with the radiation imaging device 14 when performing imaging of the radiation image, and may be a universal serial bus (USB) memory, a secure digital (SD) memory card (Registered Trademark), or the like which is detachably mounted in the radiation imaging device 14.

The I/F unit 28 has a function of performing communication of various kinds of information with the console 18 with wireless communication using electric waves or light, or the like. The radiation imaging device 14 of this embodiment performs communication with the console 18 through wireless local area network (LAN) communication. Specifically, the radiation imaging device 14 performs communication with the console 18 using wireless-fidelity (Wi-Fi).

The imaging stand 16 of this embodiment includes a stand control unit 30, a display unit drive unit 34, a display unit 36, an opening/closing sensor 38, a weight sensor 40, and an I/F unit 42, in addition to the moving unit 32 described above. The stand control unit 30, the stepping motors 33 of the moving unit 32, the display unit drive unit 34, the opening/closing sensor 38, the weight sensor 40, and the I/F unit 42 are connected to transmit and receive various kinds of information through a bus 47, such as a system bus or a control bus, to and from one another.

The stand control unit 30 has a function of controlling the overall operation of the imaging stand 16, and includes a CPU, a ROM, and a RAM. The ROM stores various processing programs, which are executed on the CPU, and the like in advance. The RAM has a function of temporarily storing various kinds of data.

As described above, the moving unit 32 has a function of moving the grid 15S for normal imaging in the longitudinal direction of the imaging stand 16 by driving the stepping motors 33 in a state of holding the grid 15S for normal imaging.

The display unit 36 of this embodiment is connected to the display unit drive unit 34, has the positioning recommended position display unit 36A and the grid position display unit 36B (also see FIG. 2), and has a function of displaying a positioning recommended position and a grid position described below in detail. In the imaging stand 16 of this embodiment, as a specific example of the positioning recommended position display unit 36A and the grid position display unit 36B, a display unit having a plurality of light emitting diodes (LEDs) arranged linearly is used. The display unit drive unit 34 has a function of controlling the display on the display unit 36.

The opening/closing sensor 38 is provided near the door 70B of the housing 70, and has a function of detecting opening/closing of the door 70B. In this embodiment, although as a specific example of the opening/closing sensor 38, a magnetic sensor is used, the invention is not limited thereto. For example, a sensor using light, such as a photointerrupter, a mechanical sensor, such as an opening/closing switch, or the like may be used as the opening/closing sensor 38.

The weight sensor 40 is provided in the holding stand 78A of the moving unit 32, and has a function of detecting the grid 15S for normal imaging being attached to the holding stands 78A by the user. In this embodiment, as a specific example of the weight sensor 40, a sensor using a strain gauge is used. Although the imaging stand 16 of this embodiment has the four holding stands 78A, the weight sensor 40 may be provided only in one of the four holding stands.

The I/F unit 42 has a function of performing communication of various kinds of information with the console 18 through wireless communication using electric waves or light, or the like. The imaging stand 16 of this embodiment performs communication with the console 18 through wireless LAN communication. Specifically, the imaging stand 16 performs communication with the console 18 using Wi-Fi.

The console 18 of this embodiment functions as a server computer. The console 18 of this embodiment has a function of controlling the moving of the grid 15S for normal imaging held by the imaging stand 16.

The console 18 includes a control unit 50 which functions as a control unit of the invention, a storage unit 52, a display unit drive unit 54, a display unit 56, an operation input detection unit 58, an operating unit 60, and an I/F unit 64. The control unit 50, the storage unit 52, the display unit drive unit 54, the operation input detection unit 58, and the I/F unit 64 are connected to transmit and receive various kinds of information through a bus 67, such as a system bus or a control bus, to and from one another.

The control unit 50 has a function of controlling the overall operation of the console 18, and includes a CPU, a ROM, a RAM, and a hard disk drive (HDD). The ROM stores various processing programs, which include a moving processing program to be executed on the CPU, and the like in advance. The RAM has a function of temporarily storing various kinds of data. The HDD has a function of storing various kinds of data.

The display unit 56 of this embodiment has a function of displaying various kinds of information relating to imaging, the radiation image, and the like. The display unit drive unit 54 has a function of controlling the display of various kinds of information on the display unit 56. The operating unit 60 is used when the user inputs information relating to imaging of a radiation image, or the like. The operating unit 60 of this embodiment includes, for example, a touch panel, a touch pen, a plurality of keys, a mouse, and the like. When the operating unit 60 is a touch panel, the operating unit 60 may be integrated with the display unit 56. The operation input detection unit 58 has a function of detecting an operation state of the operating unit 60.

The I/F unit 64 has a function of performing communication of various kinds of information between the radiation imaging device 14 and the imaging stand 16 through wireless communication using electric waves or light, or the like. The console 18 of this embodiment uses wireless LAN communication when performing communication with the radiation imaging device 14 and the imaging stand 16. Specifically, the console 18 of this embodiment performs communication with the radiation imaging device 14 and the imaging stand 16 using Wi-Fi.

The storage unit 52 stores the order information of the object W, or the like. As a specific example of the storage unit 52, an HDD, an SSD, or the like is considered.

Next, the operation of the radiation imaging system 10 of this embodiment when a radiation image is imaged will be described.

In the radiation imaging system 10 of this embodiment, when imaging of a radiation image is performed, the user selects order information corresponding to imaging to be performed from now in the console 18 and instructs to start imaging of the radiation image. In this embodiment, the console 18 acquires the order information relating to imaging of radiation images of objects of a plurality of people including the object W from an external system, such as an RIS, in advance, and stores the order information in the storage unit 52 or the like. When imaging is performed with order information unissued, for example, when additional imaging is performed, or the like, the user may input information corresponding to order information with the operating unit 60.

After the grid 15 of the type according to imaging is attached inside the imaging stand 16, the user positions the object W in front of the door 70B of the imaging stand 16. If positioning is completed, the user instructs the irradiation of radiation R according to the selected order information through the console 18. Radiation R is irradiated from the radiation irradiation device 12 in response to the instruction, and the radiation imaging device 14 is irradiated with radiation R transmitted through the object W through the door 70B of the imaging stand 16 and the grid 15. The radiation imaging device 14 generates image data of a radiation image according to irradiated radiation R and transmits image data to the console 18.

In this way, when imaging of a radiation image is performed, the console 18 performs controls relating to imaging of the radiation image by emitting radiation R from the radiation irradiation device 12 to make the radiation imaging device 14 generate image data of the radiation image, and performs control the moving of the grid 15 through the imaging stand 16.

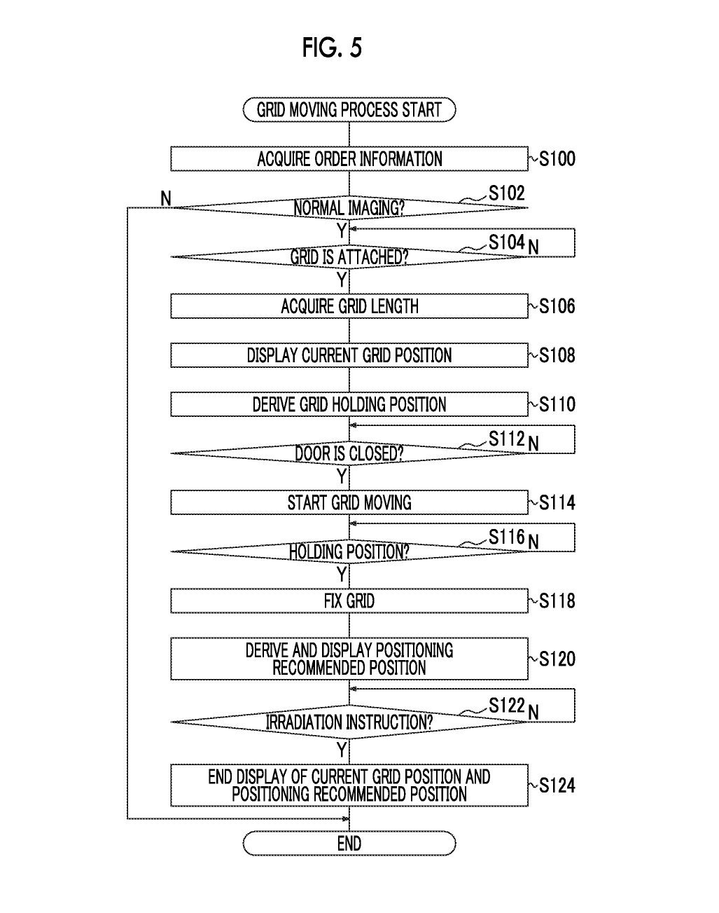

Next, the operation of the console 18 of this embodiment to control the moving of the grid 15 when performing imaging of a radiation image will be described. FIG. 5 is a flowchart showing an example of the flow of a grid moving process which is executed by the control unit 50 of the console 18 of this embodiment in this case. In the console 18 of this embodiment, the control unit 50 executes the moving processing program stored in the ROM provided therein to execute the grid moving process. This grid moving process is executed if the user instructs the start of imaging of the radiation image.

In Step S100 of FIG. 5, the control unit 50 acquires the order information selected by the user. In this embodiment, although the control unit 50 reads and acquires the order information from the storage unit 52, a way of acquiring the order information is not particularly limited. The order information may be acquired directly from an external system, and when the order information is not issued, for example, when additional imaging is performed, information corresponding to order information input from the user using the operating unit 60 may be regarded as the order information and acquired.

Next, in Step S102, the control unit 50 determines whether or not to perform normal imaging. A determination method regarding whether or not to perform normal imaging is not particularly limited, and for example, when the order information includes an instruction to the effect of performing normal imaging, it may be determined to perform normal imaging. When the order information includes an instruction to the effect of performing elongated imaging, it may be determined not to perform normal imaging. When normal imaging or elongated imaging is determined in advance according to an imaging region, and the order information includes information indicating the imaging region, it may be determined whether or not to perform normal imaging according to the imaging region included in the order information indicated by information. As a specific example, a form in which, when the imaging region is a chest, an abdomen, or the like, it is determined to perform normal imaging, and when the imaging region is a spine, a lower limb, or the like, since elongated imaging is performed, it is determined not to perform normal imaging is considered. In this way, when it is determined whether or not to perform normal imaging from the imaging region, it is desirable to consider information (for example, information (age, sex, and the like) relating to the physique of the object W) relating to the size of the imaging region, in addition to the imaging region.

When normal imaging is not performed, that is, when elongated imaging is performed, the determination in Step S102 is negative, and this grid moving process ends. In the radiation imaging system 10 of this embodiment, when elongated imaging is performed, the grid for elongated imaging is attached to the imaging stand 16 by the user. The way of attaching the grid for elongated imaging to the imaging stand 16 is not particularly limited, and similarly to the grid 15S for normal imaging, the grid for elongated imaging may be attached to the holding stands 78A by the screws 79, or may be separately fixed to the imaging stand 16 using a fixing member (not shown). When elongated imaging is performed, since the grid for elongated imaging is not moved in the longitudinal direction of the imaging stand 16, this grid moving process ends.

When normal imaging is performed, the determination in Step S102 is affirmative, and the process progresses to Step S104.

In Step S104, the control unit 50 determines whether or not the grid 15S for normal imaging is attached to the holding stands 78A of the moving unit 32 of the imaging stand 16. Specifically, the control unit 50 of this embodiment determines that the grid 15S for normal imaging is attached when the detection result representing that any object is attached is received from the imaging stand 16 compared to a state where the grid 15S for normal imaging is not attached to the holding stands 78A.

Until the grid 15S for normal imaging is attached to the holding stands 78A, the determination in Step S104 is negative and it waits for attachment, and when the grid 15S for normal imaging is attached to the holding stands 78A, the determination is affirmative and the process progresses to Step S106.

In Step S106, the control unit 50 acquires the length of the attached grid 15S for normal imaging in the longitudinal direction of the imaging stand 16 (hereinafter, simply referred to as “the length of the grid 15S for normal imaging”). A method of acquiring the length of the grid 15S for normal imaging is not particularly limited. For example, the length of the grid 15S for normal imaging is uniquely determined in advance, and when the length of the grid 15S for normal imaging is stored in the storage unit 52, the length of the grid 15S for normal imaging may be read and acquired from the storage unit 52. When the order information includes information relating to the length of the grid 15S for normal imaging, the length of the grid 15S for normal imaging may be acquired from the order information. When the user inputs the length of the grid 15S for normal imaging, the input length of the grid 15S for normal imaging may be acquired. A sensor which detects the length of the grid 15S for normal imaging may be provided in the belt 74 of the imaging stand 16 or the inner wall of the housing 70, and the length of the grid 15S for normal imaging may be acquired based on the detection result of the sensor.

Next, in Step S108, the control unit 50 displays the current position of the grid 15S for normal imaging on the imaging stand 16. Specifically, the control unit 50 of this embodiment derives the current position of the grid 15S for normal imaging using the position of the holding stands 78A, to which the grid 15S for normal imaging is attached, and the length of the grid 15S for normal imaging. An instruction to turn on an LED of the grid position display unit 36B provided at the derived current position of the grid 15S for normal imaging is transmitted to the stand control unit 30 of the imaging stand 16. The stand control unit 30 of the imaging stand 16 turns on the LED of the grid position display unit 36B based on the instruction received from the console 18. In regard to the position of the holding stands 78A, to which the grid 15S for normal imaging is attached, when the grid 15S for normal imaging is at a position determined in advance, the position determined in advance may be acquired, and when the grid 15S for normal imaging has been moved at the previous time, the position of the holding stands 78A after previous moving may be stored and the stored position may be acquired.

Next, in Step S110, the control unit 50 derives the position (hereinafter, referred to as a “holding position”) of the grid 15S for normal imaging when imaging of the radiation image indicated by the order information selected by the user is performed.

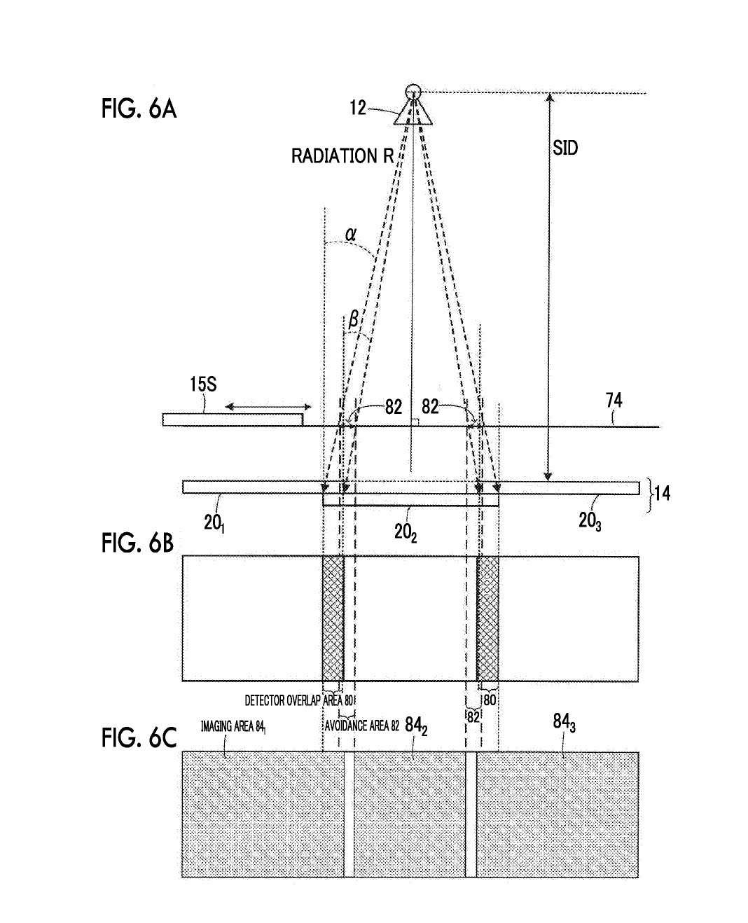

Hereinafter, in the radiation imaging system 10 of this embodiment, the holding position of the grid 15S for normal imaging will be described in detail referring to FIGS. 6A to 6C. FIGS. 6A to 6C are explanatory views illustrating the positions of the radiation irradiation device 12, the radiation imaging device 14, and the grid 15S for normal imaging of the embodiment and the irradiation state of radiation R.

In the radiation imaging system 10 of this embodiment, avoidance areas 82 which should be avoided as the holding position of the grid 15S for normal imaging are provided. Specifically, positions where positions where the end portions of adjacent radiation detectors 20 overlap each other overlap the end portion of the grid 15S for normal imaging in the incidence direction of radiation R are determined as the avoidance areas 82 of the grid 15S for normal imaging. Specifically, positions where a step image and an end image described in detail below are reflected in the radiation image generated by the radiation detector 20 are determined as the avoidance areas 82 of the grid 15S for normal imaging. The control unit 50 of the embodiment performs control for preventing the grid 15S for normal imaging from being held in the avoidance areas 82.

As shown in FIGS. 6A to 6C, the radiation imaging device 14 of this embodiment is configured such that the end portions of adjacent radiation detectors 20 overlap each other. Specifically, the end portions of the imaging surfaces (the surfaces on which pixels (not shown) effective for imaging are arranged in a matrix) of the radiation detectors 20 in the incidence direction of radiation R.

In the radiation imaging device 14 of this embodiment, the end portions of adjacent radiation detectors 20 overlap each other in consideration of variations on manufacturing of the radiation detectors 20, expansion due to the temperature of the radiation detectors 20, or the like. The range (area) of the imaging surface in the overlap portion is determined in advance according to the range of oblique incidence of radiation R irradiated from the radiation irradiation device 12, or the like.

In the radiation imaging device 14 of this embodiment, as shown in FIG. 6A, as a specific example, the end portions overlap each other in a so-called terrace shape in which the radiation detectors 20₁ and 20₃ at both ends are on an upper side (a side close to the radiation irradiation device 12), and the central radiation detector 20₂ is on a lower side (a side far from the radiation irradiation device 12).

For this reason, in the radiation detector 20₂ arranged on the lower side, a step due to the end portions of the radiation detectors 20₁ and 20₃ in the overlap portion of the radiation detectors 20₁ and 20₃ on the upper side occurs. For this reason, the shadow of the overlap portion of the radiation detectors 20₁ and 20₃ on the upper side is reflected in the radiation image generated by the radiation detector 20₂, and a step image due to the step is included.

The radiation images generated by the radiation detectors 20₁ and 20₃ arranged on the upper side are the same as a radiation image generated using a single radiation detector 20, and a step image is not included in the generated (imaged) radiation image.

Since the end portion of the grid 15S for normal imaging is projected to the radiation imaging device 14 by radiation R, an end image representing the end portion of the grid 15 is included in the radiation image generated by the radiation detector 20.

Accordingly, the step image and the end image of the grid 15S for normal imaging are included in the entire radiation image generated by the radiation imaging device 14 of this embodiment. Specifically, when a radiation image is imaged by the radiation imaging device 14, a plurality of pixels of the radiation detector 20₂ includes pixels where an electric charge by radiation R transmitted through the overlap portion is stored and pixels where an electric charge by radiation R transmitted through the end portion is stored. As described above, the step image is included in the radiation image generated by the radiation detector 20₂ on the lower side. Since the position of the end image of the grid 15S for normal imaging is determined according to the incidence angle of radiation R, it is not necessarily the case where the step image is included in the radiation image generated by the radiation detector 20₂ on the lower side. For example, when the end portion of the grid 15S for normal imaging is held at a position facing the radiation detector 20₁ or 20₃, the end image is included in the radiation image generated by the radiation detector 20₁ or 20₃. However, when the end portion of the grid 15S for normal imaging is near the step of the radiation detector 20, both the step image and the end image may be included in the radiation image generated by the radiation detector 20₂.

Accordingly, in this embodiment, the step image included in the radiation image is corrected by the control unit 50 of the console 18. Hereinafter, correction of a step image which is performed by the control unit 50 of the console 18 of this embodiment will be described. A function of correcting a step image may be provided in any device in the radiation imaging system 10, and for example, the radiation imaging device 14 may have a function of correcting a step image. In this case, image data of the radiation image with the step image corrected is transmitted from the radiation imaging device 14 to the console 18.

Since the step image is included in the radiation image generated by the radiation detector 20₂ on the lower side, correction of the step image is performed only for the radiation image generated by the radiation detector 20₂ on the lower side.

In this case, it is necessary to recognize by which of the radiation detectors 20 on the upper and lower sides the radiation image acquired from the radiation imaging device 14 by the control unit 50 is generated. The recognition method is not particularly limited. For example, information indicating whether each radiation detector 20 is a radiation detector on the upper side or the lower side may be added to image data of the radiation image and image data may be transmitted to the console 18.

When correction of the step image is performed, first, the control unit 50 detects the position of the step image from the radiation image. Since the position of the step image in the generated radiation image is different according to the incidence angle of radiation R with respect to the radiation detector 20, the control unit 50 of this embodiment detects the position of the step image from the radiation image.

A detection method of the position of the step image from the radiation image is not particularly limited. As a specific example, the control unit 50 of this embodiment detects the position of the boundary between the step image and another radiation image by detecting an image representing a line from the radiation image and detects the position of the step image based on the detected position of the boundary. Hereinafter, the boundary between the step image and another radiation image is simply referred to as a “boundary”.

A detection method of the line is not particularly limited, and a general method may be used, for example, Hough transform or the like may be used. A method of detecting the position of the step image from the position of the boundary is not particularly limited, and for example, an image from the position of the boundary to a predetermined position may be detected as a step image.

Although when the position of the boundary is detected from the radiation image, a process for detecting the position of the boundary may be performed for the entire radiation image, in this embodiment, the position of the boundary is detected by setting an area estimated to include the position of the boundary as a search range and searching only the search range. In regard to the search range, for example, a range where the position of the boundary of the step image is included in the radiation image may be obtained in advance by the design specification of the radiation imaging device 14, an experiment using an actual machine, or the like as a search range. In the control unit 50 of this embodiment, the search range is determined as detector overlap areas 80 (see FIG. 6B) where the end portions of adjacent radiation detectors 20 overlap each other. The detector overlap areas 80 are determined based on the incidence angle of radiation R, the thickness of the radiation detectors 20, the distance of the overlap portion in the incidence direction of radiation R, and the length (the length of the radiation imaging device 14 in the longitudinal direction) of the overlap portion. The detector overlap areas 80 of this embodiment are a first area and a second area of the invention.

In this way, in the radiation imaging system 10 of this embodiment, since the position of the boundary is detected only within the search range, it is possible to improve detection accuracy and to reduce a detection time compared to a case where the position of the boundary is detected for the entire radiation image.

If the position of the step image is detected, the control unit 50 corrects the detected step image. In the control unit 50 of this embodiment, the step image is corrected by performing correction to reduce the density difference between the density of the step image and the density of another radiation image.

In this way, in the correction of the step image which is performed by the control unit 50, when the position of the boundary is detected by searching the search range, if an end image due to the end portion of the grid 15S for normal imaging is reflected within the search range, the correction of the step image may not be appropriately performed. For example, while there is concern that the control unit 50 erroneously detects the end image as the position of the boundary, in this case, when erroneous detection occurs, the position of the step image is erroneously detected; thus, the correction accuracy of the step image is degraded. For this reason, image quality of the corrected radiation image is degraded.

In contrast, in the radiation imaging system 10 of this embodiment, when the grid 15S for normal imaging is held in a state where the end portion of the grid 15S for normal imaging is positioned in the avoidance area 82, the end image due to the end portion of the grid 15S for normal imaging is included in the detector overlap areas 80.

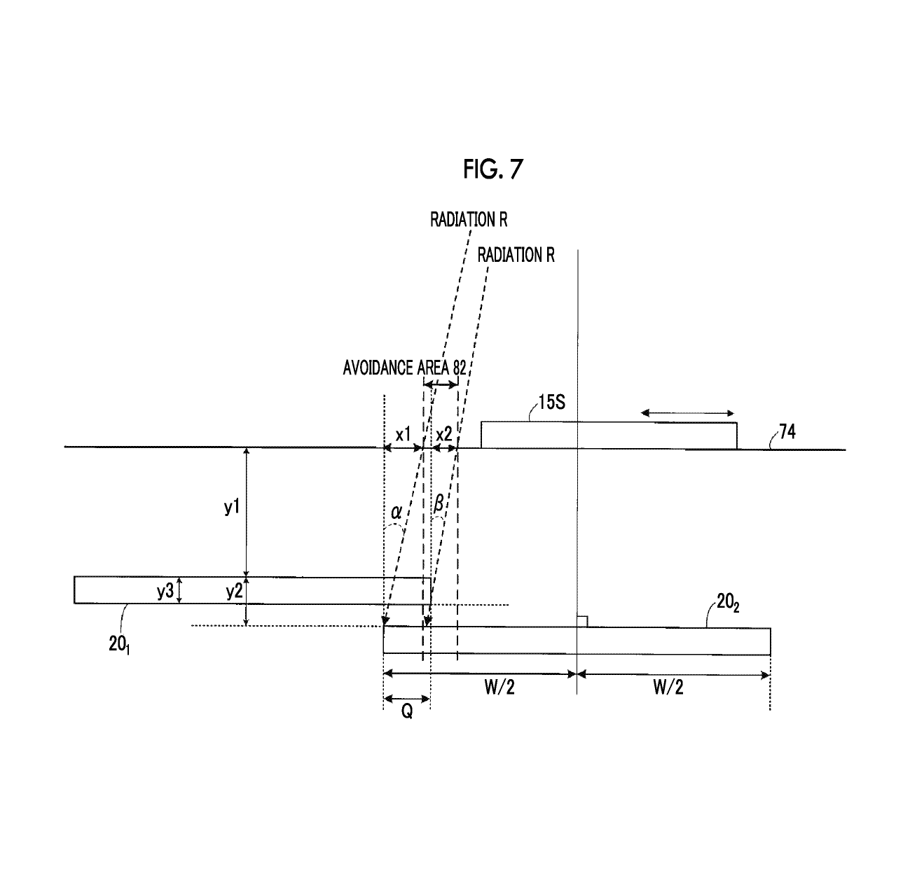

The avoidance area of the end portion of the grid 15S for normal imaging of this embodiment will be described below referring to FIG. 7, in addition to FIGS. 6A to 6C. FIG. 7 is a partial enlarged view of a portion of the avoidance area 82 of the grid 15S for normal imaging in FIGS. 6A to 6C. In FIGS. 6A to 6C, and 7, description of the holding stands 78A will be omitted.

Here, a case where the imaging region of the object W is positioned at a position corresponding to the imaging surface of the radiation detector 20₂ and normal imaging is performed is considered. In this case, in the radiation imaging system 10 of this embodiment, the radiation irradiation device 12 is arranged at a position facing the vicinity of the center of the imaging surface of the radiation detector 20₂, and irradiates radiation R from the radiation irradiation device 12 toward the object W.

As shown in FIGS. 6 and 7, when radiation R obliquely enters at an incidence angle of 90 degrees at the center of the imaging surface of the radiation detector 20₂, the incidence angle of radiation R on a side close to on the irradiation side of radiation R of the end portion of the radiation detector 20₂ is α, and the incidence angle of radiation R on a side far from the irradiation side of radiation R of the end portion of the radiation detector 20₁ overlapping the radiation detector 20₂ is β.

The distance from the belt 74 (accurately, the lower surface of the grid 15S for normal imaging) to the imaging surface of the radiation detector 20₁ in the direction perpendicular to the imaging surface is y1, and the distance from the imaging surface of the radiation detector 20₁ to the imaging surface of the radiation detector 20₂ in the direction perpendicular to the imaging surface is y2. In other words, y2 becomes a value obtained by adding the thickness of the radiation detector 20₁ and the distance between the radiation detector 20₁ and the radiation detector 20₂.

The distance (the direction in the up-down direction in FIGS. 6A to 6C) from the imaging surface of the radiation detector 20₁ to the radiation irradiation device 12 in the direction perpendicular to the imaging surface is a source image distance (SID), and the length of the radiation detector 20₂ in the longitudinal direction of the radiation imaging device 14 is W.

If the distance from the end portion of the radiation detector 20₂ shown in FIG. 7 to the avoidance area 82 in the longitudinal direction of the radiation imaging device 14 is x1, the relationship of Expressions (1) and (2) described below is obtained.

x1/(y1+y2)=tan α (1)

(W/2)/(SID+y2)=tan α (2)

The distance x1 is obtained by Expression (3) described below from Expression (1) and (2) described above.

x1=(W/2)(y1+y2)/(SID+y2) (3)

If the length of an overlap portion of the radiation detector 20₁ and the radiation detector 20₂ in the longitudinal direction of the radiation imaging device 14 is Q, the thickness of the radiation detector 20₁ is y3, and the distance from the end portion of the radiation detector 20₁ shown in FIG. 7 to the avoidance area 82 in the longitudinal direction of the radiation imaging device 14 is x2, the relationship of Expressions (4) and (5) described below is obtained.

x2/(y1+y3)=tan β (4)

(W/2−Q)/(SID+y3)=tan β (5)

The distance x2 from the end portion of the radiation detector 20₁ to the avoidance area 82 is obtained by Expression (6) described below from Expressions (4) and (5) described above.

x2=(W/2−Q)(y1+y3)/(SID+y3) (6)

Accordingly, the avoidance area 82 of the end portion of the grid 15S for normal imaging becomes an area between a position where the distance from the end portion of the radiation detector 20₂ to the avoidance area 82 in the longitudinal direction of the radiation imaging device 14 is the distance x1 and a position where the distance from the end portion of the radiation detector 20₁ in the longitudinal direction of the radiation imaging device 14 to the avoidance area 82 is the distance x2. In FIG. 7, while description is omitted, similarly, an area between a position of the distance x1 from the end portion of the radiation detector 20₂ to the avoidance area 82 and a position of the distance x2 from the end portion of the radiation detector 20₃ to the avoidance area 82 also becomes the avoidance area 82.

In this way, as described above, in the radiation imaging system 10 of this embodiment, in order to prevent an end image from being included in the vicinity of the step image included in the radiation image generated by the radiation imaging device 14, the avoidance areas 82 are provided. As described above, in the radiation imaging device 14 of this embodiment, since a step image is not included in the radiation images generated by the radiation detectors 20₁ and 20₃, an end image is not included in the vicinity of the step image in the radiation images by the radiation detectors 20₁ and 20₃. For this reason, as shown in FIG. 6C, when imaging is performed while positioning the imaging region of the object W only in an imaging area 84₁ corresponding to the imaging surface of the radiation detector 20₁ or an imaging area 84₃ corresponding to the imaging surface of the radiation detector 20₃, it is not necessary to provide the avoidance areas 82.

While a step image is included in the radiation image generated by the radiation detector 20₂, as shown in FIG. 6C, when the imaging region of the object W is positioned in the detector overlap areas 80 to be reflected only in the imaging area 84₂ where the imaging region of the object W is not reflected, it is not necessary to provide the avoidance areas 82.

For this reason, the control unit 50 of this embodiment first determines where the imaging region of the object W is positioned based on the order information or an instruction from the user or the like when the positioning position of the imaging region is instructed from the user, thereby determining whether or not it is necessary to provide the avoidance areas 82. Then, when it is determined that it is necessary to provide the avoidance areas 82, as described above, the avoidance areas 82 are derived, and a position where the end portion of the grid 15S for normal imaging is not positioned in the avoidance areas 82 is derived as the holding position of the grid 15S for normal imaging. As described above, while the avoidance areas 82 are obtained by many parameters (y1, y2, y3, SID, Q, W, and the incidence angles α, β of radiation R), when the control unit 50 cannot acquire these values, positions on the belt 74 corresponding to an area where adjacent radiation detectors 20 overlap each other may be determined as the avoidance areas 82.

In this way, in the radiation imaging system 10 of this embodiment, since the holding position (the avoidance area 82) of the grid 15S for normal imaging is determined based on the incidence angles of radiation R toward the grid 15S for normal imaging and the radiation imaging device 14 (the radiation detector 20), it is possible to prevent an end image due to the end portion of the grid 15S for normal imaging from being included within the search range of the position of the boundary in the radiation image imaged by the radiation detector 20.

With this, in the console 18 of this embodiment, since it is possible to detect the position of the boundary with excellent accuracy, it is possible to detect the position of the step image with excellent accuracy. Accordingly, it is possible to improve the accuracy of correction of the step image, and to improve image quality of a radiation image.

Next, in Step S112, the control unit 50 determines whether or not the door 70B is closed. Specifically, when a signal representing the detection result indicating the door 70B is closed is received from the imaging stand 16, the control unit 50 of this embodiment determines that the door 70B is closed. Even if a time determined in advance has elapsed after the grid 15S for normal imaging is attached to the holding stands 78A, when it is not detected that the door 70B is closed, it is desirable to perform a warning to the user using the display unit 56 or the like.

Until the door 70B is closed, the determination in Step S112 is negative and it waits for closing, and when the door 70B is closed, the determination is affirmative, and the process progresses to Step S114.

Next, in Step S114, the control unit 50 starts the moving of the grid 15S for normal imaging. Specifically, the control unit 50 instructs the imaging stand 16 to start the moving of the grid 15S for normal imaging, and instructs the holding position derived by the process in Step S110 described above. If this instruction is received, the stand control unit 30 of the imaging stand 16 drives the stepping motors 33 of the moving unit 32 to move the grid 15S for normal imaging to the instructed holding position. The stand control unit 30 of this embodiment recognizes the moving distance of the grid 15S for normal imaging by the number of steps of a drive signal of the stepping motors 33.

In this embodiment, the turning-on position of the LED of the grid position display unit 36B is changed by the stand control unit 30 of the imaging stand 16 according to the moving of the grid 15S for normal imaging.

Next, in Step S116, the control unit 50 determines whether or not the grid 15S for normal imaging reaches the holding position. If the moving of the grid 15S for normal imaging to the holding position instructed from the console 18 by the stand control unit 30 of the imaging stand 16 is completed, the imaging stand 16 of this embodiment transmits a signal representing the completion of the moving to the console 18. The control unit 50 of the console 18 determines whether or not the grid 15S for normal imaging reaches the holding position by determining whether or not this signal is received.

Until the grid 15S for normal imaging reaches the holding position, the determination in Step S116 is negative and it waits for the grid 15S for normal imaging reaching the holding position, and when the grid 15S for normal imaging reaches the holding position, the determination is affirmative and the process progresses to Step S118.

In Step S118, the control unit 50 fixes the grid 15S for normal imaging at the holding position. Specifically, the control unit 50 performs an instruction to fix the holding stands 78A to the imaging stand 16.

Next, in Step S120, the control unit 50 derives the positioning recommended position and makes the imaging stand 16 display the positioning recommended position. Specifically, the imaging stand 16 is instructed to turn on the LED of the positioning recommended position display unit 36A provided at a position corresponding to the derived positioning recommended position. The imaging stand 16 turns on the LED of the positioning recommended position display unit 36A based on the instruction received from the console 18.

In the radiation imaging system 10 of this embodiment, the positions where the grid 15S for normal imaging and the imaging areas 84₁ to 84₃ shown in FIG. 6C overlap each other are determined as the positioning recommended position. When the imaging region of the object W is positioned at the positioning recommended position, as described above, since a step image is not included in the radiation image generated by the radiation detector 20, it is possible to further improve image quality of a radiation image.

Next, in Step S122, the control unit 50 determines whether or not the irradiation of radiation R is instructed from the user. In the radiation imaging system 10 of this embodiment, as described above, the user attaches the grid 15S for normal imaging to the holding stands 78A of the imaging stand 16 and then closes the door 70B. Thereafter, the user positions the object Win front of the door 70B of the imaging stand 16. If positioning is completed, the user instructs the irradiation of radiation R through the console 18.

Accordingly, until the irradiation of radiation R is instructed, the determination in Step S122 is negative and it waits for the instruction, and if the irradiation of radiation R is instructed, the determination is affirmative and the process progresses to Step S124.

In Step S124, the control unit 50 ends the display of the current position of the grid and the positioning recommended position, and then ends this grid moving process. Specifically, the control unit 50 transmits an instruction to turn on the LEDs of the positioning recommended position display unit 36A and the grid position display unit 36B to the imaging stand 16. In the stand control unit 30 of the imaging stand 16 which receives the instruction, the LEDs of the positioning recommended position display unit 36A and the grid position display unit 36B are turned off

In this way, the radiation imaging system 10 of this embodiment includes the radiation detectors 20₁ to 20₃ which have the imaging surface, on which radiation R is incident, and generate the radiation image of an imaging target according to radiation R transmitted through an imaging region as the imaging target of the object W and incident on the imaging surface, the grid 15S for normal imaging which has an effective area narrower than the imaging surface and eliminates scattered radiation included in radiation R transmitted through the imaging target and incident on the effective area, and the moving unit 32 which functions as a holding unit capable of holding the grid 15S for normal imaging at a plurality of positions along the imaging surface where the imaging surface and the effective area overlap each other.

In this embodiment, “a plurality of positions along the imaging surface” mean a plurality of positions at predetermined intervals with respect to the imaging surface in the incidence direction of radiation R.

Accordingly, when normal imaging is performed, since it is possible to arrange the grid 15S for normal imaging at a plurality of positions according to imaging, it is possible to improve convenience for the user.

### Second Embodiment

In the first embodiment, a case where, when normal imaging is performed, the console 18 automatically moves the grid 15S for normal imaging to an appropriate holding position has been described. In contrast, in this embodiment, a case where the user moves the grid 15S for normal imaging and adjusts the position of the grid 15S for normal imaging with the console 18 will be described.

The schematic configuration (see FIG. 1) of the radiation imaging system 10 of this embodiment is the same as the first embodiment, thus, detailed description will be omitted.

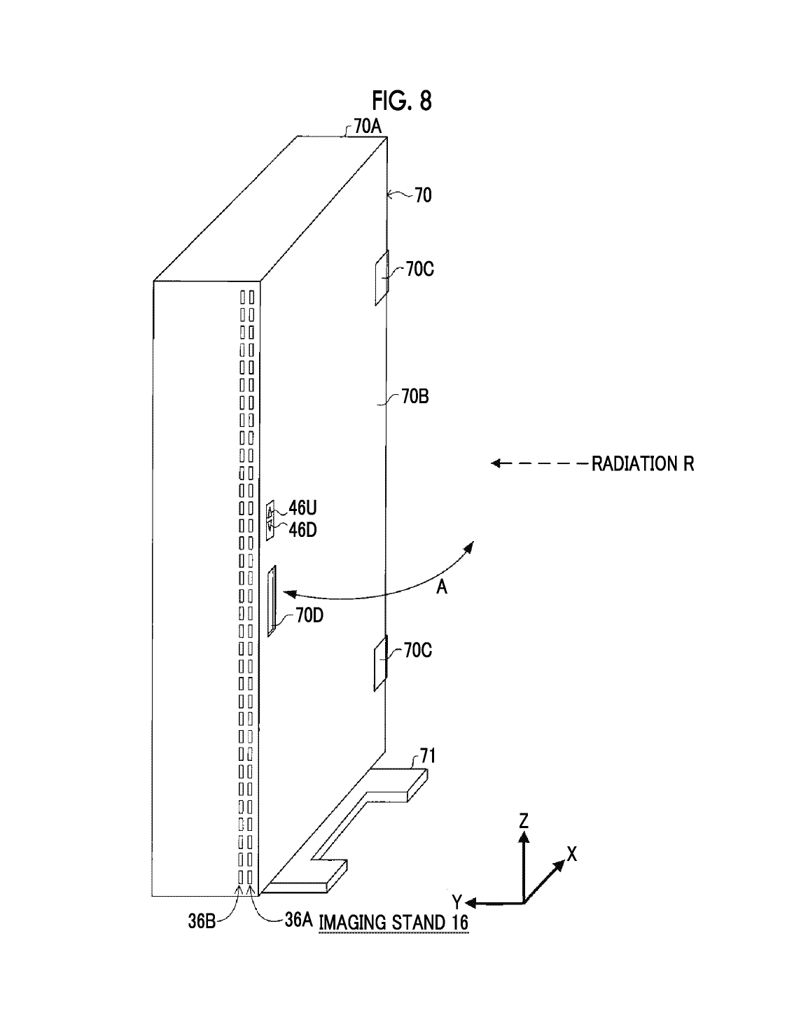

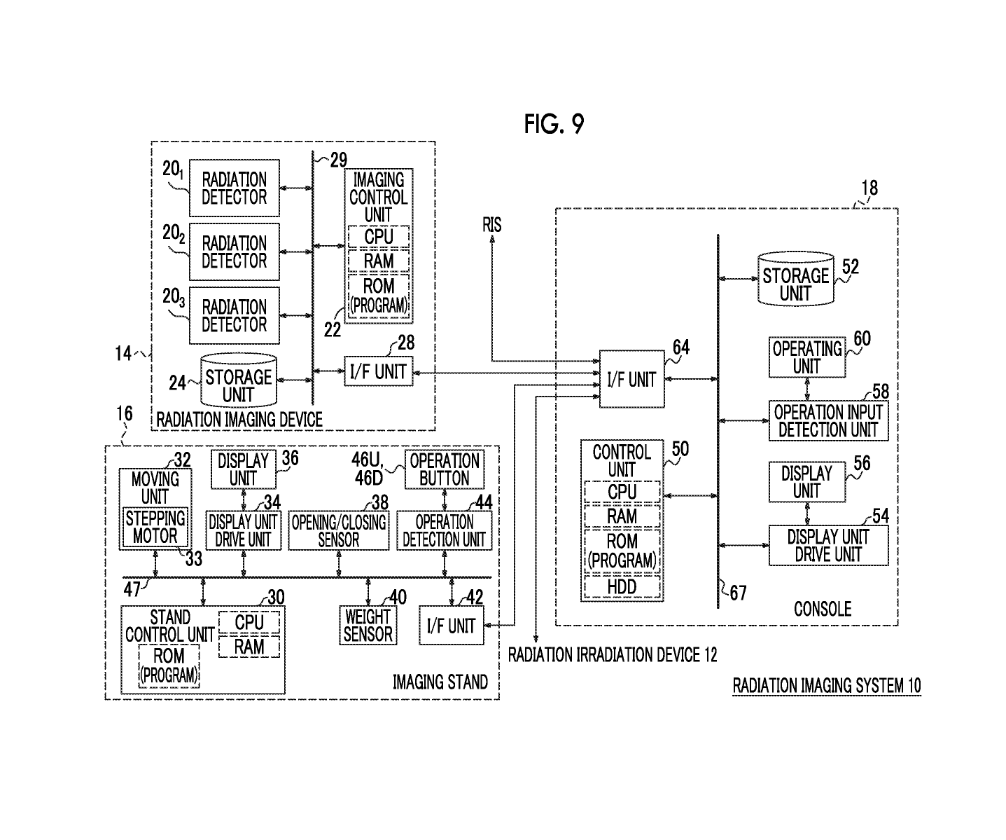

In the radiation imaging system 10 of this embodiment, since an imaging stand 16 is different from that in the first embodiment, the imaging stand 16 will be described. FIG. 8 is a perspective view of the imaging stand 16 of this embodiment when viewed from the side. FIG. 9 is a block diagram showing an example of the schematic configuration of the radiation imaging device 14, the imaging stand 16, and the console 18 of the radiation imaging system 10 of this embodiment.

As shown in FIG. 8, in the imaging stand 16 of this embodiment, the door 70B is provided with operation buttons 46U and 46D which are used when the user instructs the moving of the grid 15S for normal imaging. As shown in FIG. 9, the imaging stand 16 of this embodiment is different from the imaging stand 16 (see FIG. 4) of the first embodiment in that an operation detection unit 44 and operation buttons 46U and 46D are provided.

When moving the grid 15S for normal imaging attached to the moving unit 32 toward the upper side (a side away from the bottom plate 71 in the Z-axis direction of FIG. 8), the user operates the operation button 46U. When moving the grid 15S for normal imaging attached to the moving unit 32 toward the lower side (a side close to the bottom plate 71 in the Z-axis direction of FIG. 8), the user operates the operation button 46D. The operation detection unit 44 has a function of detecting the operation states of the operation buttons 46U and 46D. In the imaging stand 16 of this embodiment, while the user continues to operate the operation button 46U, in other words, while the operation detection unit 44 continues to detect that the operation button 46U is operated, a stand control unit 30 which functions as a reception unit of the invention continues to move the grid 15S for normal imaging toward the upper side. Similarly, while the user continues to operate the operation button 46D, in other words, while the operation detection unit 44 continues to detect that the operation button 46D is operated, the stand control unit 30 continues to move the grid 15S for normal imaging toward the lower side.

Although the positions where the operation buttons 46U and 46D are provided are not limited to this embodiment, in the imaging stand 16 of this embodiment, the door 70B is provided with the operation buttons 46U and 46D, whereby it is easy for the user to operate the operation buttons 46U and 46D in a state where the door 70B is closed compared to a state where the door 70B is open. In this way, the operation buttons 46U and 46D are easily operated in a state where the door 70B is closed compared to a case where the door 70B is open, whereby it is possible to prevent the user from coming into contact with the grid 15S for normal imaging or the moving unit 32.

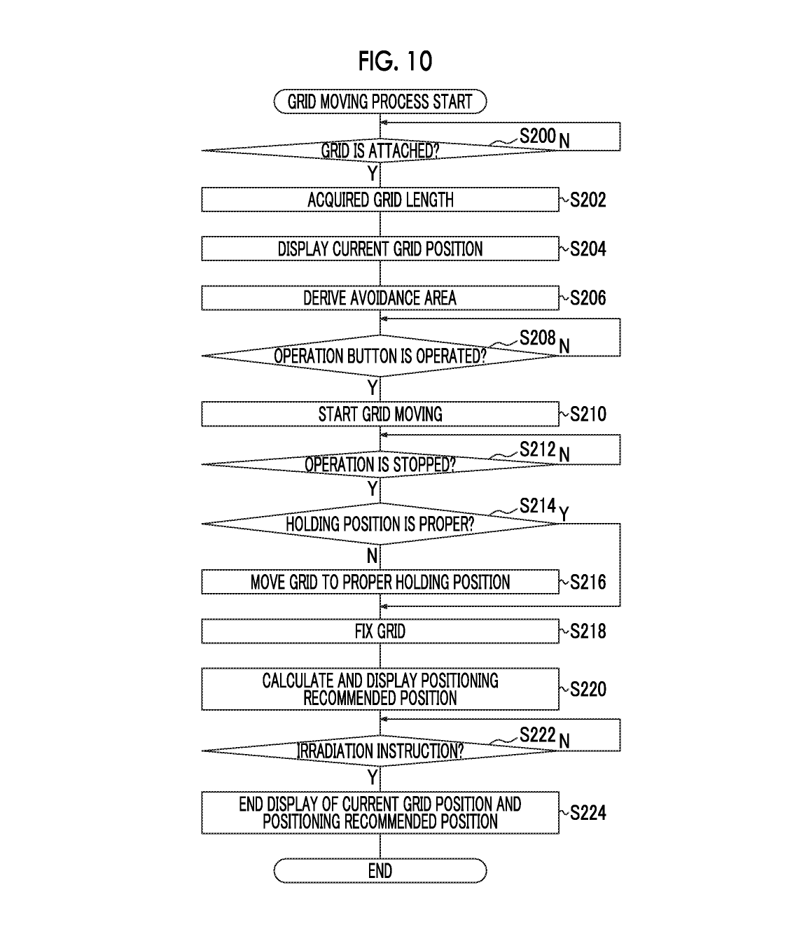

Next, a grid moving process which is executed by the control unit 50 of the console 18 of this embodiment will be described will be described. FIG. 10 is a flowchart showing an example of the flow of the grid moving process which is executed by the control unit 50 of the console 18 of this embodiment.

In FIG. 10, detailed description of steps in which the same process as in the grid moving process shown in FIG. 5 is performed will be omitted.

Each process of Steps S200 to S204 of FIG. 10 corresponds to each process of Steps S104 to S108 (see FIG. 5) of the grid moving process of the first embodiment.

In Step S200, the control unit 50 determines whether or not the grid 15S for normal imaging is attached to the holding stands 78A of the moving unit 32 of the imaging stand 16. Until the grid 15S for normal imaging is attached, the determination is negative and it waits for attachment, and when the grid 15S for normal imaging is attached, the determination is affirmative and the process progresses to Step S202.

In Step S202, the control unit 50 acquires the length of the attached grid 15S for normal imaging. Next, in Step S204, the control unit 50 makes the LED of the grid position display unit 36B of the imaging stand 16 display the current position of the grid 15S for normal imaging.

Next, in Step S206, the control unit 50 derives the avoidance areas 82 as described in the first embodiment. As described above, in the derivation of the avoidance areas 82, a plurality of parameters (y1, y2, y3, SID, Q, W, and the incidence angles α, β of radiation R) are required. For this reason, the control unit 50 of this embodiment acquires necessary parameters from the order information, information stored in the storage unit 52 in advance, or the like and derives the avoidance areas 82.

Next, in Step S208, the control unit 50 determines whether or not the operation button 46U or the operation button 46D is operated. As described above, in the radiation imaging system 10 of this embodiment, first, the user operates the operation button 46U or 46D of the imaging stand 16 to move the grid 15S for normal imaging to a desired position. When it is detected that the operation button 46U or the operation button 46D is operated, the operation detection unit 44 of the imaging stand 16 transmits a signal, which includes information relating to which of the operation buttons 46U and 46D is operated and represents that the operation is detected, to the console 18. When this signal is received, the control unit 50 of the console 18 determines that the operation button 46U or the operation button 46D is operated.

Until the operation button 46U or the operation button 46D is operated, the determination in Step S208 is negative and it waits for the operation, and when the operation button 46U or the operation button 46D is operated, the process progresses to Step S210.

In Step S210, the control unit 50 starts the moving of the grid 15S for normal imaging as in Step S114 (see FIG. 5) of the grid moving process of the first embodiment. In this embodiment, the stand control unit 30 changes the turning-on position of the LED of the grid position display unit 36B according to the moving of the grid 15S for normal imaging. For this reason, the user understands the position of the grid 15S for normal imaging from the outside of the imaging stand 16 even in a state where the door 70B is closed and the inside of the imaging stand 16 is not viewed.

In this embodiment, as in the grid moving process (see Step S112 of FIG. 5) of the first embodiment, when the door 70B is closed, the grid 15S may be moved.

Next, in Step S212, the control unit 50 determines whether or not the operation of the operation button 46U or the operation button 46D is stopped. While the control unit 50 receives a signal, which represents that the operation of the operation button 46U or the operation button 46D is detected, from the imaging stand 16, the determination in Step S212 is negative and it waits for stopping of the operation, and when the signal is not received, the determination is affirmative and the process progresses to Step S214.

In Step S214, the control unit 50 determines whether or not the grid 15S for normal imaging is held at an appropriate holding position. Specifically, the control unit 50 derives the position of the end portion of the grid 15S for normal imaging from the initial position (the position where moving starts) of the grid 15S for normal imaging, the length of the grid 15S for normal imaging, and the moving distance, and determines whether or not the end portion of the grid 15S for normal imaging enters the avoidance areas 82 derived in Step S206. An acquisition method of the moving distance of the grid 15S for normal imaging for use in deriving the position of the end portion in the control unit 50 is not particularly limited. For example, a signal representing the number of steps of the drive signal of the stepping motors 33 required for the moving of the grid 15S for normal imaging may be transmitted from the imaging stand 16 to the console 18, and the control unit 50 which receives the signal may acquire the moving distance based on the number of steps. Further, the moving distance may be acquired based on the time when the signal representing that the operation button 46U or 46D is operated continues to be received.

When the grid 15S for normal imaging is held at an appropriate holding position, the determination in Step S214 is affirmative and the process progresses to Step S218. When the grid 15S for normal imaging is not at an appropriate holding position, the determination is negative and the process progresses to Step S216.

In Step S216, the control unit 50 moves the grid 15S for normal imaging to an appropriate holding position. Specifically, the imaging stand 16 is instructed to move the grid 15S for normal imaging to a position where the position of the end portion of the grid 15S for normal imaging is outside the avoidance areas 82.

Each process of subsequent Steps S218 to S224 corresponds to each process of Steps S118 to S124 (see FIG. 5) of the grid moving process of the first embodiment.

In Step S218, the control unit 50 fixes the grid 15S for normal imaging at the holding position. Next, in Step S220, the control unit 50 derives the positioning recommended position and displays the positioning recommended position on the imaging stand 16. Next, in Step S222, the control unit 50 determines whether or not the irradiation of radiation R is instructed. The determination in Step S222 is negative, it waits for the instruction, and when determination is affirmative, the process progresses to Step S224. In Step S224, the control unit 50 ends the display of the current grid position and the positioning recommended position and then ends this grid moving process.

### Third Embodiment

In the respective embodiments described above, the console 18 performs control for determining the holding position of the grid 15S for normal imaging to an appropriate position. In contrast, in this embodiment, a case where the user attaches the grid 15S for normal imaging at a desired position and the console 18 does not perform the moving of the grid 15S for normal imaging will be described.

The schematic configuration (see FIG. 1) of the radiation imaging system 10 of this embodiment is the same as in the first embodiment, thus, detailed description will be omitted.

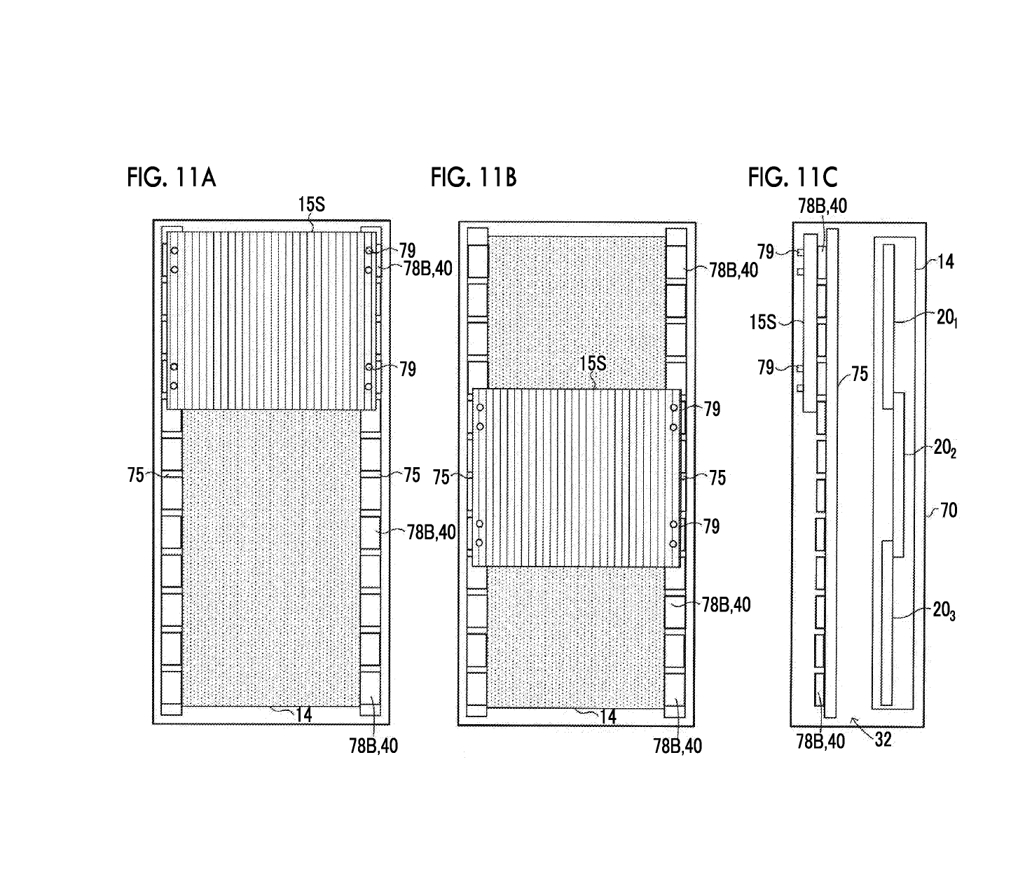

In the radiation imaging system 10 of this embodiment, since the imaging stand 16 is different from that in the first embodiment, the imaging stand 16 will be described. FIGS. 11A to 11C are a front view and a side view of the imaging stand 16 of this embodiment when viewed from the irradiation side of radiation R. FIGS. 11A and 11B are front views of a state where the radiation imaging device 14 and the grid 15S for normal imaging are housed. FIG. 11C is a side view of a state where the radiation imaging device 14 and the grid 15S for normal imaging are housed.

As shown in FIGS. 11A to 11C, the imaging stand 16 of this embodiment includes a pair of fixing units 75 which are provided in the longitudinal direction of the imaging stand 16 (radiation imaging device 14), instead of the moving unit 32 (see FIGS. 3A and 3B) in the imaging stand 16 of the respective embodiments described above. On the irradiation side of radiation R of the fixing units 75, a plurality of holding stands 78B are fixed, instead of the holding stands 78A (see FIGS. 3A and 3B) in the imaging stand 16 of the respective embodiments described above. Each of the holding stand 78B fixed to one fixing unit 75 of a pair of fixing units 75 is provided with a weight sensor 40. In this embodiment, the imaging stand 16 or the console 18 can recognize the current position of the grid 15S for normal imaging from the position of the holding stand 78B where the weight sensor 40 which detects that the grid 15S is attached is provided.

As shown in FIGS. 11A and 11B, the grid 15S for normal imaging is fixed to the holding stand 78B according to a desired position among a plurality of holding stands 78B by the screws 79, whereby in the imaging stand 16 of this embodiment, the grid 15S for normal imaging can be held at a plurality of positions.

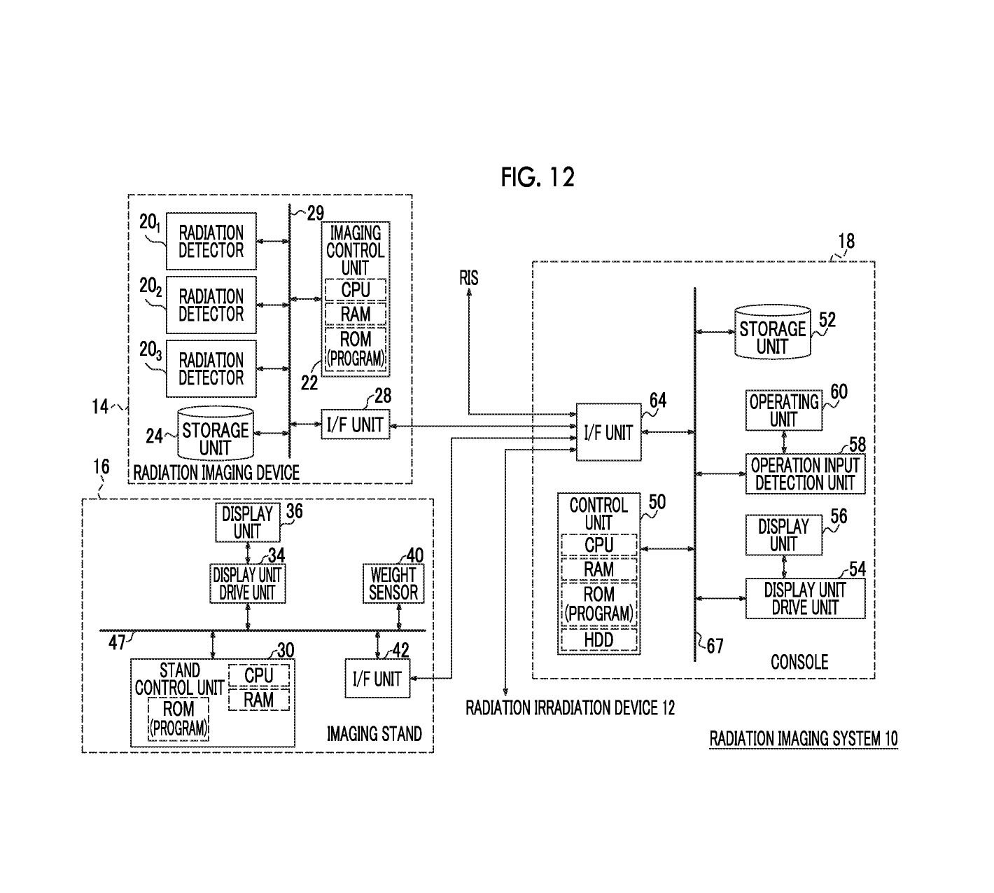

FIG. 12 is a block diagram showing the schematic configuration of the radiation imaging device 14, the imaging stand 16, and the console 18 of the radiation imaging system 10 of this embodiment.

As shown in FIG. 12, the imaging stand 16 of this embodiment is different from the imaging stand 16 (see FIG. 4) of the first embodiment in that the moving unit 32 (the stepping motors 33) and the opening/closing sensor 38 are not provided. As described above, in this embodiment, since the moving of the grid 15S for normal imaging is not performed, a configuration (the moving unit 32) for automatically moving the grid 15S for normal imaging is not required in the imaging stand 16. Furthermore, in this embodiment, since the grid 15S for normal imaging is not moved, the opening/closing sensor 38 which is used to prevent the user from coming into contact with the grid 15S for normal imaging being moved is not required.

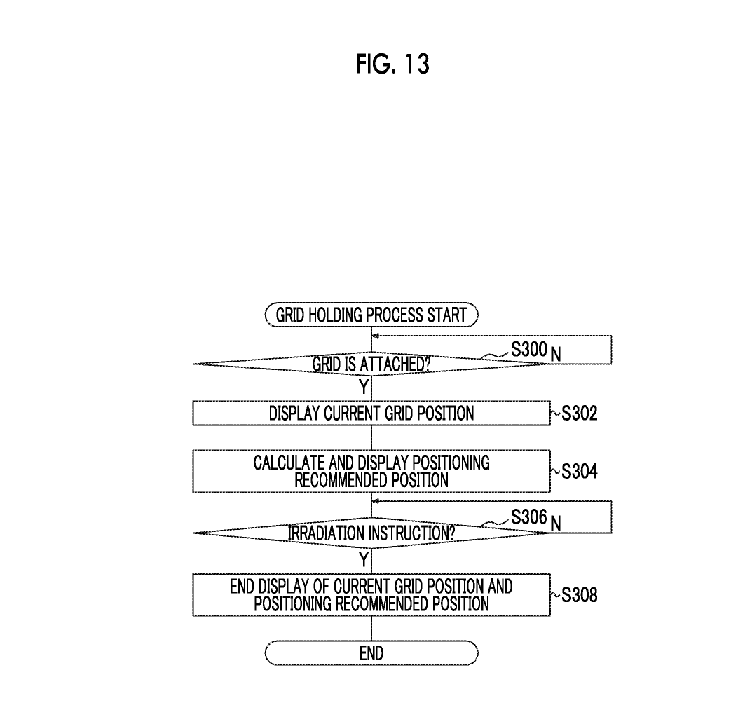

Next, the operation of the console 18 of this embodiment will be described. The console 18 of this embodiment executes a grid holding process, instead of the grid moving process (see FIG. 5 or 10) which is executed by the console 18 of the respective embodiments described above. FIG. 13 is a flowchart showing an example of the flow of the grid holding process which is executed by the control unit 50 of the console 18 of this embodiment.

The grid holding process shown in FIG. 10 includes the same process as the grid moving process shown in FIG. 5, thus, detailed description of the steps in which the same process is performed will be omitted.

Each process of Steps S300, S302, and S304 to S308 of FIG. 13 corresponds to each process of Steps S104, S108, and S120 to S124 (see FIG. 5) of the grid moving process of the first embodiment.

In Step S300, the control unit 50 determines whether or not the grid 15S for normal imaging is attached to the holding stands 78B of the moving unit 32 of the imaging stand 16. Until the grid 15S for normal imaging is attached, the determination is negative and it waits for the attachment, and when the grid 15S for normal imaging is attached, the determination is affirmative and the process progresses to Step S302.

In Step S302, the control unit 50 makes the LED of the grid position display unit 36B of the imaging stand 16 display the current position of the grid 15S for normal imaging.

Next, in Step S304, the control unit 50 derives the positioning recommended position and displays the positioning recommended position on the imaging stand 16. Next, in Step S306, the control unit 50 determines whether or not the irradiation of radiation R is instructed. When the determination in Step S306 is negative, it waits for the instruction, and when the determination is affirmative, the process progresses to Step S308. In Step S308, the control unit 50 ends the display of the current grid position and the positioning recommended position and then ends this grid moving process.

In this embodiment, as in the respective embodiments described above, the control unit 50 of the console 18 may derive the avoidance areas 82 or an appropriate holding position of the grid 15S for normal imaging and may provide the avoidance areas 82 or the appropriate holding position of the grid 15S for normal imaging to the user. For example, the appropriate holding position may be provided to the user by turning on the LED of the grid position display unit 36B. Furthermore, for example, when the position where the user attaches the grid 15S for normal imaging is not an appropriate holding position, a warning determined in advance may be performed. A warning method is not particularly limited, and for example, the control unit 50 may function as a warning unit, and may display, on the display unit 56, a warning sentence indicating that the grid 15S for normal imaging is not at an appropriate holding position, or may turn on the LED of the grid position display unit 36B.

### Fourth Embodiment

In the respective embodiments described above, a case where elongated imaging is performed using the grid for elongated imaging has been described. In contrast, in this embodiment, a case where elongated imaging is performed using the grid 15S for normal imaging will be described.

In order to appropriately eliminate scattered radiation with the grid 15, it is desirable to perform imaging using the grid 15 of the type according to the amount of scattered radiation or the like. The amount of scattered radiation changes with the physique of the object W, the irradiation amount of radiation R, or the like. For this reason, in order to appropriately eliminate scattered radiation, it is preferable to prepare a plurality of types of grids 15.

In general, it may not be preferable practically that a plurality of types of grids for elongated imaging more expensive than the grid 15S for normal imaging are prepared. For this reason, in the radiation imaging system 10 of this embodiment, when the grid 15S for normal imaging which can appropriately eliminate scattered radiation is provided, elongated imaging can be performed using the grid 15S for normal imaging, instead of the grid for elongated imaging.

Specifically, in a state where the imaging surface of the radiation imaging device 14 having an elongated imaging surface and the effective area of the grid 15S for normal imaging, the grid 15S for normal imaging is moved to a plurality of positions in the longitudinal direction of the radiation imaging device 14, and the radiation images generated by the radiation detectors 20 with the irradiation of radiation R for each moved position are composed, whereby elongated imaging can be performed in the same manner as so-called divided imaging.

The configuration (see FIGS. 1 and 2) of the radiation imaging system 10 of this embodiment is the same as in the first embodiment, thus, detailed description thereof will be omitted.

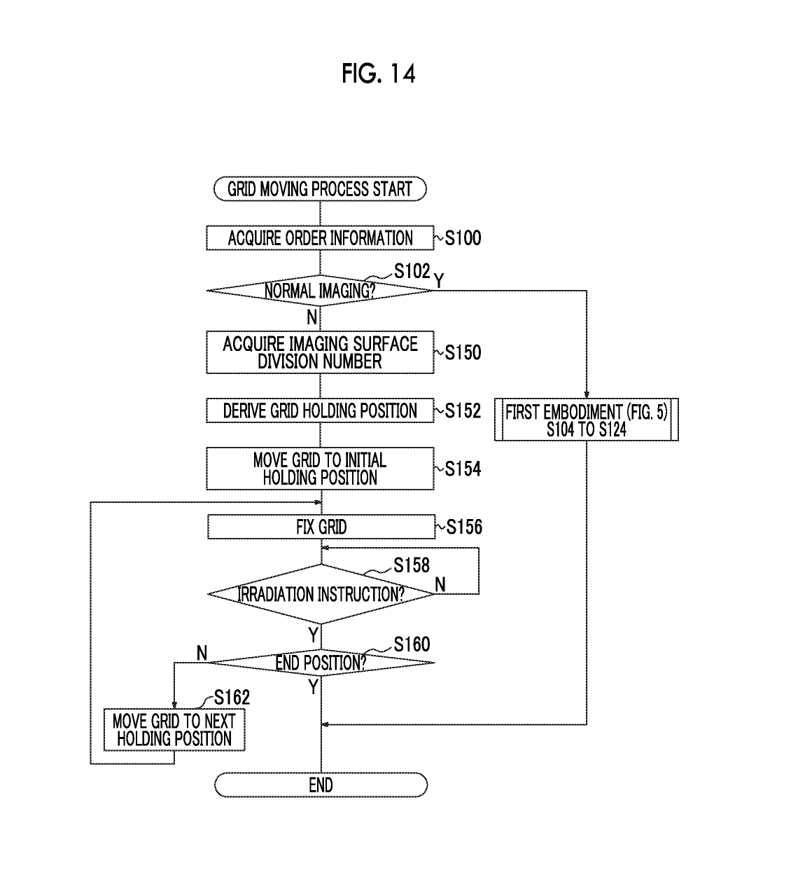

A grid moving process which is executed by the control unit 50 of the console 18 of this embodiment will be described. FIG. 14 is a flowchart showing an example of the flow of the grid moving process which is executed by the control unit 50 of the console 18 of this embodiment.

In FIG. 14, steps in which the same process as in the grid moving process shown in FIG. 5 is performed are represented by the same reference numerals, and detailed description of the same process will be omitted.

As shown in FIG. 14, when the determination in Step S102 is affirmative (when it is determined to be normal imaging), the same processes as in Steps S104 to S124 (see FIG. 5) of the grid moving process of the first embodiment are performed, and then, this grid moving process ends.

When the determination in Step S102 is negative, the process progresses to Step S150. As described above, when the determination in Step S102 is negative, this means that elongated imaging is performed.

In Step S150, the control unit 50 acquires the number of divisions of the imaging surface. As described above, in this embodiment, in order to perform elongated imaging in the same manner as divided imaging, the number of divisions is acquired. The number of divisions is not particularly limited, and may be a number according to the length of the radiation detector 20 in the longitudinal direction of the radiation imaging device 14 and the length of the grid 15S for normal imaging, may be determined in advance, and may be derived by the control unit 50 during this grid moving process. As described above, in order to make determine the holding position of the grid 15S for normal imaging to an appropriate position, it is desirable to increase the number of divisions more than the number of radiation detectors 20. In this embodiment, since the number of radiation detectors 20 is three, the number of divisions is preferably set to be equal to or greater than four.

Next, in Step S152, the control unit 50 derives the holding position of the grid 15S for normal imaging. Specifically, the holding position of the grid 15S for normal imaging is derived for each divided area.

Next, in Step S154, the control unit 50 instructs the imaging stand 16 to move the grid 15S for normal imaging to the initial position of the holding position. The initial position is the holding position of the grid 15S for normal imaging corresponding to the position of the divided area where imaging is initially performed.

Next, in Step S156, the control unit 50 fixes the grid 15S for normal imaging at the holding position as in Step S118 (see FIG. 5) of the grid moving process of the first embodiment.

Next, in Step S158, the control unit 50 determines whether or not the irradiation of radiation R is instructed as in Step S122 (see FIG. 5) of the grid moving process of the first embodiment. When the determination in Step S158 is negative, it waits for the instruction, and when the determination is affirmative, the process progresses to Step S160. The radiation detectors 20 generate image data of the radiation images according to irradiated radiation R. Generated image data of the radiation images may be sequentially transmitted from the radiation imaging device 14 to the console 18, or may be stored in the storage unit 24 once and may be collectively transmitted to the console 18 after image data of the radiation images of all divided areas are generated.

In Step S160, the control unit 50 determines whether or not the holding position of the grid 15S for normal imaging is an end position. When the holding position of the grid 15S for normal imaging is not the end position, the determination in Step S160 is negative and the process progresses to Step S162. The end position is the holding position of the grid 15S for normal imaging corresponding to the divided area where imaging is performed last.

In Step S162, the control unit 50 instructs to move the grid 15S for normal imaging to the next holding position, then returns to Step S156, and repeats the processes of Steps S156 to S160.

When the determination in Step S160 is affirmative, this grid moving process ends.

In the radiation imaging system 10 of this embodiment, although elongated imaging is performed in the same manner as divided imaging, and imaging is performed using the grid 15S for normal imaging for each divided area of the imaging surface, the radiation images are generated by all radiation detectors 20 of the radiation imaging device 14 regardless of the divided areas. For this reason, it is desirable to associate information representing the position of the divided area with the generated radiation images.

The console 18 cuts a radiation image corresponding to the divided area from the radiation images generated by the radiation detectors 20 (trimming or the like) and composes the radiation images of the divided areas to generate an elongated radiation image.

In this way, in the radiation imaging system 10 of the embodiment, since the grid 15S for normal imaging can be held at a plurality of positions, it is possible to perform elongated imaging even using the grid 15S for normal imaging.

As described above, the radiation imaging system 10 of the respective embodiments described above includes the radiation detectors 20₁ to 20₃ which have the imaging surface, on which radiation R is incident, and generate the radiation image of an imaging target according to radiation R transmitted through an imaging region as the imaging target of the object W and incident on the imaging surface, the grid 15S for normal imaging which has an effective area narrower than the imaging surface and eliminates scattered radiation included in radiation R transmitted through the imaging target and incident on the effective area, and the moving unit 32 which functions as a holding unit capable of holding the grid 15S for normal imaging at a plurality of positions along the imaging surface where the imaging surface and the effective area overlap each other.

In the radiation imaging system 10 of this embodiment, in this way, since the grid 15S for normal imaging can be held at a plurality of positions along the imaging surface, it is possible to perform normal imaging using the radiation imaging device 14 and the grid 15S for normal imaging.

With this, it is possible to perform normal imaging using the radiation imaging device 14 without using the grid 15 for elongated imaging. Accordingly, convenience for the user is improved. Furthermore, in the radiation imaging system 10 of this embodiment, there is no need for arranging a plurality of types of expensive grids 15 for elongated imaging in advance.

In the radiation imaging system 10 of this embodiment, since the grid 15S for normal imaging can be held at a position corresponding to the position of the imaging region of the object W, the imaging region of the object W is positioned at a desired position of the user.

Furthermore, a device, such as the console 18, which performs an image process of a radiation image can recognize the holding position of the grid 15S for normal imaging, it is possible to recognize an area where the imaging region is imaged from the position of the grid 15S for normal imaging; therefore, it is possible to perform an image process (for example, a trimming process) according to the area where the imaging region is imaged with excellent accuracy.

In the respective embodiments described above, during normal imaging, when moving the radiation irradiation device 12, for example, when moving the radiation irradiation device 12 according to the position where the imaging region of the object W is positioned, the radiation irradiation device 12 may be moved in conjunction with the grid 15S for normal imaging. For example, the moving unit 32 of the imaging stand 16 may move the grid 15S for normal imaging according to the moving amount of the radiation irradiation device 12 by the user. Conversely, for example, the console 18 may move the radiation irradiation device 12 according to the moving amount of the grid 15S for normal imaging.

In the radiation imaging device 14 of the respective embodiments described above, although the radiation detectors 20₁ and 20₃ are provided on a side close to the radiation irradiation device 12, and the radiation detector 20₂ is provided on a side far from the radiation irradiation device 12, the arrangement of the radiation detectors 20 is not limited to this embodiment. For example, the radiation detectors 20₁ and 20₃ may be provided on a side far from the radiation irradiation device 12, and the radiation detector 20₂ may be provided on a side close to the radiation irradiation device 12. Furthermore, the radiation detectors 20 may be arranged stepwise, and for example, the radiation detector 20₁ may be arranged on a side closest to the radiation irradiation device 12, and the radiation detector 20₃ may be arranged on a side farthest from the radiation irradiation device 12.

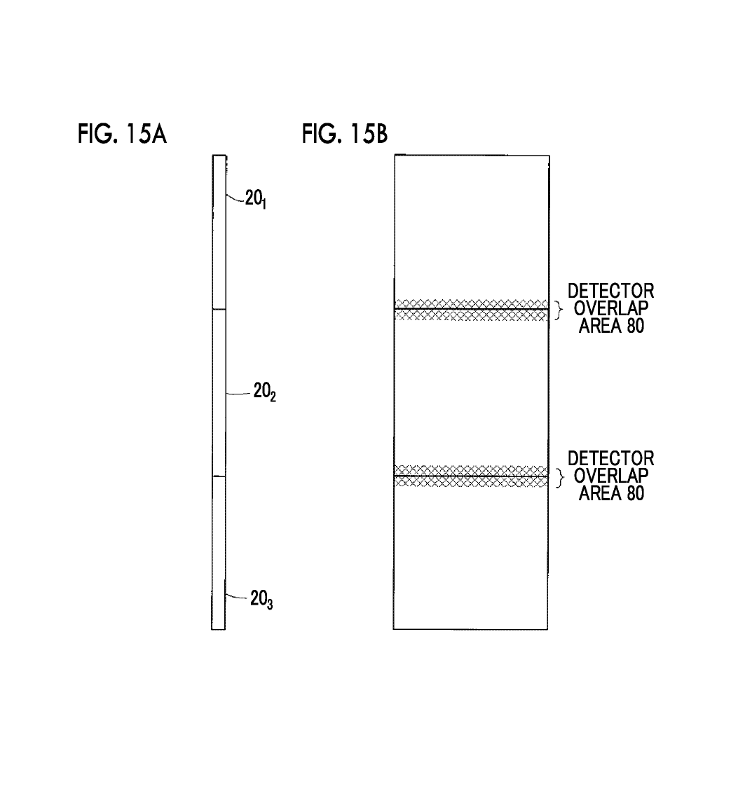

In the respective embodiments described above, although a case where a plurality of radiation detectors 20 are arranged such that the end portions of adjacent radiation detectors 20 overlap each other in the incidence direction of radiation R has been described, the arrangement of the radiation detectors 20 is not limited thereto. For example, as shown in FIGS. 15A and 15B, the radiation detectors 20 may be arranged in parallel while aligning the height of the imaging surface. FIGS. 15A and 15B show a side view of the radiation detector 20 in this case and a front view of the radiation detectors 20 when viewed from the irradiation side of radiation R. As shown in FIG. 15A, instead of overlapping the end portions of adjacent radiation detectors 20 each other in the incidence direction of radiation R, the radiation detectors 20 may be arranged in parallel while aligning the height of the imaging surface. In this case, as shown in FIG. 15B, an area according to a distance determined in advance by an experiment or the like from the end portions where the radiation detectors 20 are in contact with each other may be set as the detector overlap area 80.

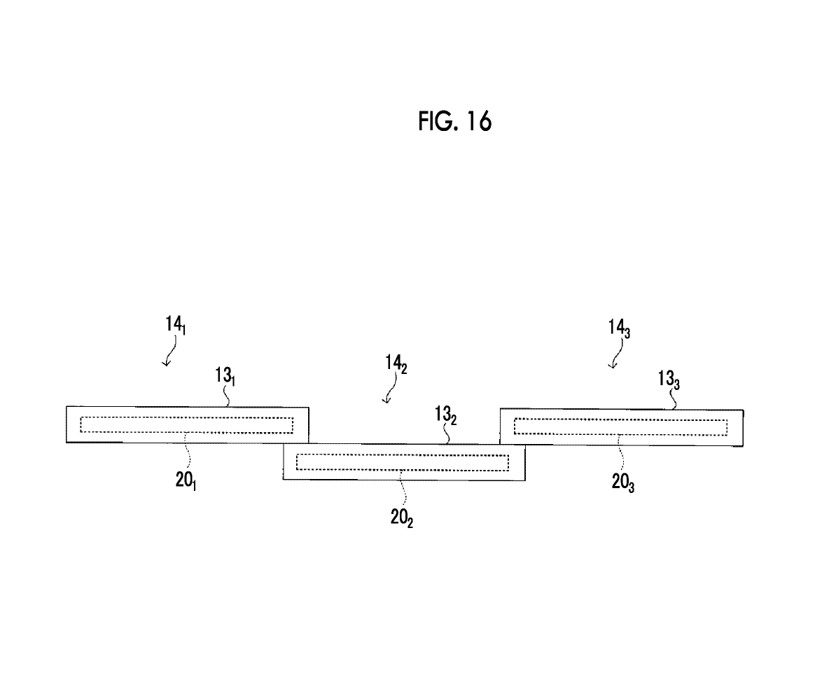

In the respective embodiments described above, although a case where the radiation imaging system 10 includes one radiation imaging device 14 including a plurality of radiation detectors 20 in the housing 13 has been described, the invention is not limited thereto, and the number of radiation detectors 20, the number of radiation imaging devices 14, and the arrangement of the radiation detectors 20 are not particularly limited. As a specific example, FIG. 16 shows a side view in a case where the radiation imaging system 10 includes a plurality of radiation imaging devices 14 including one radiation detector 20. FIG. 16 shows a case where a radiation imaging device 141 including a radiation detector 20₁ in a housing 131, a radiation imaging device 142 including a radiation detector 20₂ in a housing 132, and a radiation imaging device 143 including a radiation detector 20₃ in a housing 133 are alternately arranged. As shown in FIG. 16, a plurality of radiation imaging devices 14 (here, as a specific example, three) including one radiation detector 20 in the housing 13 may be provided.

In the imaging stand 16 of the respective embodiments described above, although the door 70B is provided on the irradiation side of radiation R has been described, the position of the door 70B is not limited thereto. For example, the door 70B may be provided on a side surface (in FIG. 2, a surface on which the positioning recommended position display unit 36A and the grid position display unit 36B are provided) of the housing 70, and the grid 15 may be inserted from the side surface and attached.

In the respective embodiments described above, although a case where the imaging stand 16 includes the moving unit 32 has been described, the moving unit 32 may be provided other than the imaging stand 16. For example, a moving device which has a function as the moving unit 32 may be provided separately from the imaging stand 16. Furthermore, for example, the radiation imaging device 14 may have a part or all of the functions of the moving unit 32.

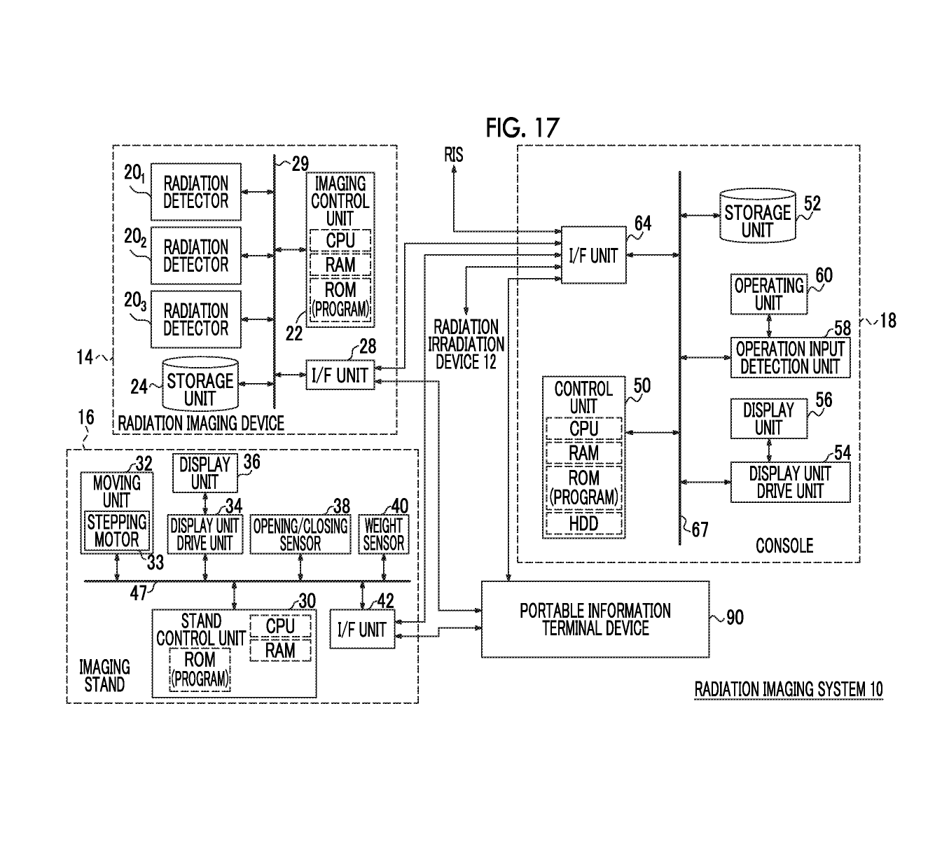

In the respective embodiments, although a case where the console 18 has a function as a control unit controlling the moving of the grid 15S for normal imaging has been described, the function of the control unit may be provided in other devices. For example, the imaging stand 16 may have a function as a control unit. When the radiation imaging system 10 includes a control device assisting the console 18, another control device may have a function as a control unit controlling the moving of the grid 15S for normal imaging. FIG. 17 is a block diagram showing an example of the schematic configuration of the radiation imaging system 10 including a portable information terminal device 90 as such a control device. As the portable information terminal device 90, for example, devices which can be driven with an internal battery, specifically, a tablet terminal device, a smartphone which is a so-called personal digital assistant (PDA), or the like is considered.

The object W may not be a person, and may be a living thing, such as animal or plant, or other objects.

Radiation R of the respective embodiments described above is not particularly limited, and X-rays, γ-rays, or the like can be applied.

The configuration and operation of the radiation imaging system 10, the radiation imaging device 14, the imaging stand 16, the console 18, and the like described in the respective embodiments described above are merely examples, and obviously various modifications can be made within the scope without departing from the spirit of the invention.

What is claimed is:

1. A radiation imaging system comprising:
a radiation detector which has an imaging surface, on which radiation is incident, and generates a radiation image of an imaging target according to radiation transmitted through the imaging target and incident on the imaging surface;
a grid which has an effective area narrower than the imaging surface and eliminates scattered radiation included in radiation transmitted through the imaging target and incident on the effective area; and
a holding unit which is able to hold the grid at a plurality of positions along the imaging surface where the imaging surface and the effective area overlap each other.

2. A radiation imaging system comprising:
a radiation detector group which includes a plurality of radiation detectors configured to have an imaging surface, on which radiation is incident, and to generate a radiation image of an imaging target according to radiation transmitted through the imaging target and incident on the imaging surface, adjacent radiation detectors being arranged in parallel in a direction intersecting an incidence direction of radiation and the end portions of the imaging surfaces of adjacent radiation detectors overlapping each other in the incidence direction of radiation;
a grid which has an effective area narrower than the imaging surface by the radiation detector group and eliminates scattered radiation included in radiation transmitted through the imaging target and incident on the effective area; and
a holding unit which is able to hold the grid at a plurality of positions along the imaging surface where a first area where the end portions of adjacent radiation detectors overlap each other and the end portion of the grid are separated in the intersection direction, and the imaging surface and the effective area overlap each other.

3. The radiation imaging system according to claim 2, further comprising:
a warning unit which performs a warning determined in advance in the case where the end portion of the grid is held at a position overlapping the first area in the intersection direction.

4. A radiation imaging system comprising:
a radiation detector group which includes a plurality of radiation detectors configured to have an imaging surface, on which radiation is incident, and to generate a radiation image of an imaging target according to radiation transmitted through the imaging target and incident on the imaging surface, adjacent radiation detectors being arranged in parallel in a direction intersecting an incidence direction of radiation, and the end portions of the imaging surfaces of adjacent radiation detectors overlapping each other in the incidence direction of radiation;
a grid which has an effective area narrower than the imaging surface by the radiation detector group and eliminates scattered radiation included in radiation transmitted through the imaging target and incident on the effective area; and
a holding unit which is able to hold the grid at a plurality of positions along the imaging surface where a second area in the radiation images generated by the radiation detector group where a step image due to a step with respect to the incidence direction in the end portions of the radiation detectors is included and an end image due to the end portion of the grid are separated, and the imaging surface and the effective area overlap each other.

5. The radiation imaging system according to claim 4, further comprising:
a warning unit which performs a warning determined in advance in the case where the grid is held at a position in the second area where the end image is included.

6. The radiation imaging system according to claim 4,
wherein the position of the second area and the position of the end image are determined based on the incidence angles of radiation with respect to the grid and the radiation detectors.

7. The radiation imaging system according to claim 5,
wherein the position of the second area and the position of the end image are determined based on the incidence angles of radiation with respect to the grid and the radiation detectors.

8. The radiation imaging system according to claim 1,
wherein the holding unit includes a moving unit which is able to move the grid in a direction along the imaging surface.

9. The radiation imaging system according to claim 2,
wherein the holding unit includes a moving unit which is able to move the grid in a direction along the imaging surface.

10. The radiation imaging system according to claim 3,
wherein the holding unit includes a moving unit which is able to move the grid in a direction along the imaging surface.

11. The radiation imaging system according to claim 4,
wherein the holding unit includes a moving unit which is able to move the grid in a direction along the imaging surface.

12. The radiation imaging system according to claim 5,
wherein the holding unit includes a moving unit which is able to move the grid in a direction along the imaging surface.

13. The radiation imaging system according to claim 8, further comprising:
a reception unit which receives an instruction to move the grid,
wherein the moving unit moves the grid based on the instruction received by the reception unit.

14. The radiation imaging system according to claim 8,
wherein the holding unit includes a control unit which controls the moving of the grid by the moving unit.

15. The radiation imaging system according to claim 14, further comprising:
an imaging stand which has the holding unit and a shield unit capable of shielding the grid from the imaging target,
wherein the control unit moves the grid with the moving unit in the case where the shield unit shields the grid from the imaging target.

16. The radiation imaging system according to claim 8, further comprising:
a radiation irradiation device which irradiates radiation,
wherein the radiation irradiation device moves in a direction intersecting the incidence direction of radiation in conjunction with the moving of the grid by the moving unit.

17. An imaging stand using the radiation imaging system according to claim 1 comprising:
a housing unit which houses a radiation detector configured to have an imaging surface, on which radiation is incident, and to generate a radiation image of an imaging target according to radiation transmitted through the imaging target and incident on the imaging surface, and a grid configured to have an effective area narrower than the imaging surface and to eliminate scattered radiation included in radiation transmitted through the imaging target and incident on the effective area; and
a holding unit which is able to hold the grid at a plurality of positions along the imaging surface where the imaging surface and the effective area overlap each other.

18. The imaging stand according to claim 17,
wherein the holding unit includes a moving unit which is able to move the grid in a direction along the imaging surface.

19. An imaging method using the radiation imaging system according to claim 1,
wherein, in the case where a radiation image is imaged using a radiation detector group which includes a plurality of radiation detectors configured to have an imaging surface, on which radiation is incident, and to generate a radiation image of an imaging target according to radiation transmitted through the imaging target and incident on the imaging surface, adjacent radiation detectors being arranged in parallel in a direction intersecting an incidence direction of radiation and the end portions of the imaging surfaces of adjacent radiation detectors overlapping each other in the incidence direction of radiation, and a grid which has an effective area narrower than the imaging surface by the radiation detector group and eliminates scattered radiation included in radiation transmitted through the imaging target and incident on the effective area,
the imaging method comprises a step of:
causing a holding unit capable of holding the grid to hold the grid at a plurality of positions along the imaging surface where a first area where the end portions of adjacent radiation detectors overlap each other and the end portion of the grid are separated in the intersection direction, and the imaging surface and the effective area overlap each other, and
causing a control unit to perform moving control for moving the grid to any of the plurality of positions by a moving unit capable of moving the grid in a direction along the imaging surface.

20. An imaging method using the radiation imaging system according to claim 1,
wherein, in the case where a radiation image is imaged using a radiation detector group which includes a plurality of radiation detectors configured to have an imaging surface, on which radiation is incident, and to generate a radiation image of an imaging target according to radiation transmitted through the imaging target and incident on the imaging surface, adjacent radiation detectors being arranged in parallel in a direction intersecting an incidence direction of radiation and the end portions of the imaging surfaces of adjacent radiation detectors overlapping each other in the incidence direction of radiation, and a grid which has an effective area narrower than the imaging surface by the radiation detector group and eliminates scattered radiation included in radiation transmitted through the imaging target and incident on the effective area,
the imaging method comprises a step of:
causing a holding unit capable of holding the grid to hold the grid at a plurality of positions along the imaging surface where an end image due to the end portion of the grid is not included in a second area in the radiation images generated by the radiation detector group where a step image due to a step with respect to the incidence direction in the end portions of the radiation detectors is included, and the imaging surface and the effective area overlap each other, and
causing a control unit to perform moving control for moving the grid to any of the plurality of positions by a moving unit capable of moving the grid in a direction along the imaging surface.
